SEQUENCE LISTING (45 sequences)

SEQ ID NO: 1 - DNA, 25 bp, Artificial Sequence
  Synthetic nucleotide
ttggttgtgtaactgatcagtaggc

SEQ ID NO: 2 - DNA, 23 bp, Artificial sequence
  Synthetic nucleotide
tggagagctagcaggaaactagg

SEQ ID NO: 3 - DNA, 48 bp, Artificial sequence
  Synthetic nucleotide
acgcaggatccgccaccatgagtctcctcaaagagcggaagccaaaaa

SEQ ID NO: 4 - DNA, 43 bp, Artificial sequence
  Synthetic nucleotide
acgcagcggccgcggggacacccgggccctccttcgtagtgtg

SEQ ID NO: 5 - RNA, 21 nt, Artificial sequence
  Synthetic nucleotide
guagauucauauuguaagguu

SEQ ID NO: 6 - RNA, 19 nt, Artificial sequence
  Synthetic nucleotide
ccaccagaguuuccacaua

SEQ ID NO: 7 - DNA, 20 bp, Artificial sequence
  Synthetic nucleotide
gcaattattccccatgaacg

SEQ ID NO: 8 - DNA, 20 bp, Artificial sequence
  Synthetic nucleotide
ggcctcactaaaccatccaa

SEQ ID NO: 9 - DNA, 18 bp, Artificial sequence
  Synthetic nucleotide
agctcgcctgagtgtgac

SEQ ID NO: 10 - DNA, 18 bp, Artificial sequence
  Synthetic nucleotide
tgcagactctggcctgta

SEQ ID NO: 11 - DNA, 17 bp, Artificial sequence
  Synthetic nucleotide
gctgcttcctgaaccac

SEQ ID NO: 12 - DNA, 18 bp, Artificial sequence
  Synthetic nucleotide
gggaactcatgcatcaag

SEQ ID NO: 13 - DNA, 21 bp, Artificial sequence
  Synthetic nucleotide
ggactctccaccacactgatg

SEQ ID NO: 14 - DNA, 21 bp, Artificial sequence
  Synthetic nucleotide
ctgctgctgctggtaagacat

SEQ ID NO: 15 - DNA, 19 bp, Artificial sequence
  Synthetic nucleotide
ccaggctacacaaagctac

SEQ ID NO: 16 - DNA, 19 bp, Artificial sequence
  Synthetic nucleotide
gcttaattcaggggcttac

SEQ ID NO: 17 - DNA, 18 bp, Artificial sequence
  Synthetic nucleotide
aaagcacctagagcaccc

SEQ ID NO: 18 - DNA, 19 bp, Artificial sequence
  Synthetic nucleotide
ggttgaatgtcttggacct

SEQ ID NO: 19 - DNA, 20 bp, Artificial sequence
  Synthetic nucleotide
ctctgtctgcggctactctg

SEQ ID NO: 20 - DNA, 20 bp, Artificial sequence
  Synthetic nucleotide
cacgaggtctgagtgacctg

SEQ ID NO: 21 - DNA, 20 bp, Artificial sequence
  Synthetic nucleotide
gcaagtgctccaatcttgca

SEQ ID NO: 22 - DNA, 20 bp, Artificial sequence
  Synthetic nucleotide
cttctctgggttggcacaca

SEQ ID NO: 23 - DNA, 25 bp, Artificial sequence
  Synthetic nucleotide
tgtttgtcactcgaaggaaccgcca

SEQ ID NO: 24 - DNA, 22 bp, Artificial sequence
  Synthetic nucleotide
tggtgctgctgtctccacttgc

SEQ ID NO: 25 - DNA, 21 bp, Artificial sequence
  Synthetic nucleotide
cgaaaagcggtagggcacgga

SEQ ID NO: 26 - DNA, 21 bp, Artificial sequence
  Synthetic nucleotide
gcctctcgtacatacagacgc

SEQ ID NO: 27 - DNA, 21 bp, Artificial sequence
  Synthetic nucleotide
ccagttctgctttggatcagc

SEQ ID NO: 28 - DNA, 23 bp, Artificial sequence
  Synthetic nucleotide
taccatgaggtcacttcagatgc

SEQ ID NO: 29 - DNA, 20 bp, Artificial sequence
  Synthetic nucleotide
gcactctcggcctacattgg

SEQ ID NO: 30 - DNA, 20 bp, Artificial sequence
  Synthetic nucleotide
cagtcccagtgacgctagac

SEQ ID NO: 31 - DNA, 20 bp, Artificial sequence
  Synthetic nucleotide
acccactagagcggtgagag

SEQ ID NO: 32 - DNA, 21 bp, Artificial sequence
  Synthetic nucleotide
acggatggggctaacttacaa

SEQ ID NO: 33 - DNA, 21 bp, Artificial sequence
  Synthetic nucleotide
agtcctcgatttgctcgaact

SEQ ID NO: 34 - DNA, 20 bp, Artificial sequence
  Synthetic nucleotide
gcatcccgaagcgaagtctt

SEQ ID NO: 35 - DNA, 21 bp, Artificial sequence
  Synthetic nucleotide
gggctgggacagtccattcta

SEQ ID NO: 36 - DNA, 20 bp, Artificial sequence
  Synthetic nucleotide
gctgctttgcctacctctcc

SEQ ID NO: 37 - DNA, 21 bp, Artificial sequence
  Synthetic nucleotide
tcgagtgacaaacacgactgc

SEQ ID NO: 38 - DNA, 19 bp, Artificial sequence
  Synthetic nucleotide
tgggccatgctccctagaa

SEQ ID NO: 39 - DNA, 23 bp, Artificial sequence
  Synthetic nucleotide
ggtgaggacaaagagtatgtctg

SEQ ID NO: 40 - DNA, 21 bp, Artificial sequence
  Synthetic nucleotide
cagcagctctctcggaatctc

SEQ ID NO: 41 - DNA, 23 bp, Artificial sequence
  Synthetic nucleotide
tggatacggggcacattattttt

SEQ ID NO: 42 - DNA, 22 bp, Artificial sequence
  Synthetic nucleotide
gaaatgccaccttttgacagtg

SEQ ID NO: 43 - DNA, 21 bp, Artificial sequence
  Synthetic nucleotide
tggatgctctcatcaggacag

SEQ ID NO: 44 - DNA, 21 bp, Artificial sequence
  Synthetic nucleotide
gcagcatcaccttcgcttaga

SEQ ID NO: 45 - DNA, 22 bp, Artificial sequence
  Synthetic nucleotide
cagatattggcatgggagcaag

(19) United States
(12) Patent Application Publication
(10) Pub. No.: US 2019/0289834 A1
(43) Pub. Date: Sep. 26, 2019

(54) ANIMAL MODELS FOR PSORIASIS AND SCREENING METHODS

(71) Applicant: U.S. GOVERMENT AS REPRESENTED BY THE DEPARTMENT OF VETERANS AFFIRS, NW Washington, DC (US)

(72) Inventors: M. Peter Marinkovich, Redwood City, CA (US); Carl Gustaf Maarten Winge, Mountain View, CA (US)

(73) Assignee: U.S. GOVERMENT AS REPRESENTED BY THE DEPARTMENT OF VETERANS AFFIRS, Washington, DC (US)

(21) Appl. No.: 16/302,325

(22) PCT Filed: May 19, 2017
(86) PCT No.: PCT/US2017/033609

§ 371 Date: Nov. 16, 2018

Related U.S. Application Data
(60) Provisional application No. 62/339,720, filed on May 20, 2016.

(51) Int. Cl. A01K 67/027; A61K 49/00

(52) U.S. Cl. CPC A01K 67/0275; A01K 2227/105; A01K 2267/0387; A61K 49/0008

(57) ABSTRACT

A biologically relevant animal model for psoriasis is provided. Epidermal-immune interactions governing epidermal tissue homeostasis are altered in psoriasis, an inflammatory disease affecting one in thirty adults. Here, we characterize Rac1 as a key mediator of this process. Rac1 activation was consistently elevated in psoriatic epidermis and primary psoriatic human keratinocytes (PHKC).

### CROSS REFERENCE

This application claims benefit and is a 371 application and claims the benefit of PCT Application No. PCT/US2017/033609, filed May 19, 2017, which claims benefit of U.S. Provisional Patent Application No. 62/339,720, filed May 20, 2016, which applications are incorporated herein by reference in their entirety.

### GOVERNMENT SUPPORT

This invention was made with Government support under contract AR047223 awarded by the National Institutes of Health. The Government has certain rights in the invention.

### BACKGROUND OF THE INVENTION

Psoriasis is an immune-mediated skin disease appearing in a chronic recurring manner. Prevalence estimates show that it affects 1-2% of the worldwide population with equal gender distribution. Psoriasis can emerge at any time of life and it usually peaks between the ages of 30-39 and 60-69. Sufferers may experience itch, pain, and/or psoriasis-related nail disease and arthritis. Significant morbidity extends to the psychosocial impact on the individual. Psoriatic patients are often stigmatized by people staring at their disfigured skin; they may have low self-esteem and would face difficulties in relationships and employment. Psoriasis has also been associated with an increased risk of cardiovascular diseases, stroke and cancer.

Histological assessment of psoriatic plaques demonstrates keratinocyte hyperproliferation with parakeratosis, epidermal elongation or rete ridges, increased angiogenesis, and dermal infiltration of inflammatory cells, including T cells, neutrophils, macrophages, and dendritic cells (DCs). Other histological features often observed in psoriatic skin include micropustules of Kogoj, microabscesses of Munro, thinned or absent granular layer, thinned suprapapillary plates, and the papillary dermis containing dilated superficial vessels.

The etiology of psoriasis is multifactorial. Environmental triggers, such as trauma, stress, infections, and drugs, activate in predisposed individuals an exaggerated inflammatory response in the skin. Although psoriasis is a disease of dysfunctional proliferation and differentiation of the keratinocytes, there is significant T cell involvement through the release of inflammatory cytokines that promote further recruitment of immune cells, keratinocyte proliferation, and sustained chronic inflammation. These T-cells proliferate in the epidermis of psoriatic plaques.

The presence of innate immune cells and their products in psoriatic skin plaques indicates a role for innate immunity. Cells of the innate immune system include macrophages, NK and NKT cells, and DCs. There is an increased number of plasmacytoid and myeloid DCs in psoriatic skin compared with non-lesional skin. Other cellular elements of innate immunity are also involved in the development of psoriasis, including high numbers of macrophages which can secrete IL-6, IL-12, IL-23, and TNF-α. Keratinocytes are also capable resident antigen-presenting cells (APCs) in the skin. When stimulated they produce large amounts of cytokines (e.g., TNF-α, IL-6, and IL-18), chemotactic chemokines (e.g., IL-8 and CCL20), and antimicrobial peptides (e.g., β-defensin and LL37).

Genome wide scans have reported at least nine chromosomal loci linked to psoriasis. PSORS1 accounts for 35-50% of the heritability of the disease. PSORS1 is located on the major histological complex (MHC) region of chromosome 6 (6p21). Three genes contained within this region are associated with psoriasis, namely, HLA-Cw6, CCHCR1 (coiled-coil α-helical rod protein), and CDSN (corneodesmosin). Other susceptibility loci have been identified which include genes expressed in keratinocytes (LCE3B (late cornified envelope 3B) and LCE3C1 (late cornified envelope 3C1)) and immune cells (IL-12B, IL23R, and IL23A), and they are involved in maintaining epidermal skin barrier and immune responses against pathogens.

Currently the first line of treatment for psoriasis is the use of topical agents. When topical therapy fails, escalated treatment often includes phototherapy, oral systemic agents, and/or injectable biological therapies. Corticosteroids, vitamin D analogues, and tazarotene all are used in the treatment of chronic plaque psoriasis. However, prolonged exposure to topical corticosteroids may lead to atrophy of the skin, permanent striae, and telangiectasia. Vitamin D analogues (e.g., calcitriol, calcipotriol, and tacalcitol) are effective antipsoriatic agents, but excessive use can lead to hypercalcaemia. The probability of treatment success doubles when combining vitamin D analogues with topical corticosteroids as compared with the vitamin D analogue monotherapy. As a result, the currently recommended first-line induction treatment of plaque psoriasis is a combination of a vitamin D analogue and a topical steroid.

Other topical agents are commonly combined with topical corticosteroids and vitamin D analogues when treating psoriatic plaques. Salicylic acid is a topical keratolytic agent used adjunctly for removing scales, and it acts by reducing coherence between keratinocytes, increasing hydration, and softening of the stratum corneum by decreasing the skin pH, however, systemic salicylic acid toxicity can occur after long-term use over large skin areas. Retinoids, another popular treatment agent for psoriasis, act on skin by mediating cell differentiation and proliferation. Systemic retinoids, e.g. Tazarotene, are associated with several adverse effects including teratogenicity, serum lipid elevations, mucocutaneous toxicity, skeletal changes, and hair loss.

Ultraviolet (UV) light therapy induces T-lymphocyte apoptosis in psoriatic lesions of the dermis and epidermis. Oral 8-methoxypsoralen-UV-A (PUVA) and narrowband UVB (NB-UVB) are well-established and effective treatments for chronic plaque psoriasis. PUVA has a response rate of approximately 80% compared with 70% for NB-UVB, however, NB-UVB is preferred because of higher convenience, except in case of very thick plaques.

Systemic treatments are often used in combination with topical therapy and phototherapy for patients with severe psoriasis. Oral systemic agents for the treatment of psoriasis include methotrexate, cyclosporine, and acitretin. Injectable biological therapies are emerging approaches for the treatment of psoriasis by targeting molecules in the inflammatory pathways. They are considered for patients with severe psoriasis that are resistant to oral immunosuppressants and phototherapy. The two major therapeutic classes of injectable biological therapies include anti-cytokine therapies and T-cell-targeted therapies. The first class consists of injectable immunoglobulins (Ig), infliximab, and adalimumab, target soluble and membrane-bound TNF-α. Other anti-cytokine therapies include Etanercept and Ustekinumab. A second therapeutic class of injectable therapies include agents that bind to T cells and prevent T-cells activation, including alefacept and efalizumab.

Dermatologists and patients would benefit from new therapies for psoriasis, particularly those that can be delivered topically.

### SUMMARY OF THE INVENTION

Rac1 is shown herein to be a key mediator of epidermal-immune interactions governing epidermal tissue homeostasis and psoriasis. Rac1 activation was consistently elevated in psoriatic epidermis and primary psoriatic human keratinocytes (PHKC). Mice expressing K14 driven V12Rac1 activated mutant closely mimicked human psoriasis, requiring an intact immune system for disease progression. Mouse and human psoriatic skin showed similar Rac1 dependent signaling and transcriptional overlap of epidermal and immune pathways. PHKC displayed Rac1-dependent upregulation of pro-inflammatory cytokines following immunocyte coculture, mimicked by overexpressing V12Rac1 in normal human keratinocytes. Modulating Rac1 activity perturbed differentiation, proliferation and inflammatory pathways including STAT3, NFκB and ZNF750. Reconstructed patient PHKC/immunocyte xenografts showed psoriasiform hyperplasia and inflammation in vivo, which was abolished by inhibiting Rac1 activity in PHKC. Rac1 is a therapeutic psoriasis target and a key orchestrator of pathologic epidermal-immune interactions. Animal models and screening methods for psoriasis are provided herein.

In some embodiments, animal models of psoriasis are provided. A transgenic mouse model for epidermal expression of activated Rac1 is provided. The transgenic animal displays features of human psoriasis, with disease activity in the skin, nails and joints closely mimicking human psoriasis clinically and histologically. Localized skin erythema and scaling, Auspitz sign, Koebnerization, response to cyclosporine and topical corticosteroids as well as pattern of arthritis closely mimic human psoriasis. The condition is requires immunocompetence. In some embodiments, the activated Rac1 gene is V12Rac1. In some embodiments, expression is driven by a keratin promoter.

In other embodiments a xenograft animal model is provided, which reproduces the psoriatic hyperplasia/inflammation seen with full thickness human psoriasis skin/PBMC xenografts while allowing genetic manipulation of epidermal cells prior to xenografting. Primary keratinocytes and fibroblasts are from human control or human psoriatic non-lesional skin, seeded on devitalized dermis and grown at the airfluid interphase prior to being xenografted to immunodeficient mice. Autologous PBMCs are injected intradermally. The keratinocytes may be genetically modified, e.g. to introduce an exogenous, activated form of Rac1 on an expression vector, operably linked to a promoter active in keratinocytes. Alternatively the keratinocytes may be treated to activate endogenous Rac1.

Screening methods are provided for candidate agents in the treatment of psoriasis, where the activity of the agent in suppressing activation of Rac1 is determined. In some embodiments the methods comprise contacting an animal model of the invention with a candidate agent, and determining the effect on the psoriasis phenotype of the animal, e.g. the transgenic or xenograft model, e.g. on skin inflammation, patterns of arthritis, immune component, lesions, etc. In some embodiments the contacting is topical.

The present invention provides methods and compositions for treating psoriasis, e.g., chronic psoriasis, targeting Rac1. The flare of psoriasis may be indicated by loss of a Psoriasis Area and Severity Index (PASI) 90 response, by loss of a Psoriasis Area and Severity Index (PASI) 75 response, by loss of a Psoriasis Area and Severity Index (PASI) 50 response, or by loss of a clear or minimal Physician's Global Assessment (PGA) rating. The loss of a PASI response may be loss of PASI response of a single body region, loss of PASI response of two body regions, loss of PASI response of three body regions, or loss of PASI response of four body regions. The body region may be trunk, lower extremities, upper extremities, or head and neck.

In one embodiment, the psoriasis is chronic psoriasis. In one embodiment, the psoriasis is plaque psoriasis, e.g., chronic plaque psoriasis. In another embodiment, the psoriasis is chronic psoriasis, e.g., chronic plaque psoriasis. In yet another embodiment, the psoriasis is moderate to severe psoriasis, e.g., moderate to severe plaque psoriasis, moderate to severe chronic psoriasis or moderate to severe chronic plaque psoriasis. In one embodiment, the subject has had a clinical diagnosis of psoriasis for at least 6 months. In another embodiment, the subject has had stable plaque psoriasis for at least 2 months.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1A-1P: An epidermal intrinsic defect of Rac1 hyperactivation in human psoriasis. (FIG. 1A) Human psoriatic lesional (n=19), (FIG. 1B) non-lesional (n=4) or (FIG. 1C) control skin (n=10) and (FIG. 1F) basal- (n=3) or (FIG. 1G) squamous cell carcinoma (n=3) was examined by confocal microscopy (IDIF) using a Rac1-GTP specific mAb. Rac1GTP-red, type VII collagen-green. (FIG. 1D,1E) Rac1GTP was significantly elevated in psoriatic lesional suprabasal and basal epidermis and in basal nonlesional psoriatic epidermis compared to control skin. (FIG. 1H) PHKC (n=7) and V12Rac1KC showed elevated, cytosolic Rac1 activation compared to NHKC by confocal microscopy. Rac1GTP-red DNA blue. (FIG. 1I,1M) EGF, (FIG. 1J,1N) TNFα, (FIG. 1K,1O) IL17A/F and (FIG. 1L,1P) IL22 trigged Rac1 hyperactivation after 90 minutes following cytokine stimulation, assayed by Rac1GTP pulldown. Unpaired t-test. *P<0.05. Error bars SEM. n=3 (TNFα, IL17), n=4 (EGF) or n=2 (IL22) per condition (PSO/CTL). PHKC/PSO: primary human psoriatic keratinocytes. NHKC/CTL: primary normal human keratinocytes. Rac1KC/V12: V12Rac1 overexpressing NHKC. Col7: type VII collagen. Scalebars 50/10 μm.

FIG. 2A-2R: (FIG. 2A-2C) Generation of K14 driven V12Rac1 activated mutant in transgenic mice demonstrating Rac1 hyperactivation by immunoblot (V12Rac1 band, upper arrow) and IDIF (Rac1GTP-red, DNA blue). (FIG. 2D) Localized skin erythema and scaling was apparent by 7 days, (FIG. 2E) progressing by 14 days. Rac1 mice were smaller compared to wild type siblings. (FIG. 2F-2I) By one month, lesions commonly localized to ears, paws, tail and snout. (FIG. 2J) Lesions showed hemorrhage (Auspitz sign) following removal of scale and (FIG. 2K,2L) trauma related lesion development resembled the Koebner phenomenon of human psoriasis. (FIG. 2M,2N) Potent topical corticosteroids improved skin lesions. (FIG. 20-2R) By one month, erythema and edema of the tail and limbs around the distal joints and paws was frequent, and ˜50% showed a pronounced mutilating arthropathy, associated with bony deformations of the paws.

FIG. 3A-3S: Epidermal Rac1 activation, in the presence of a normal immune system, produces a psoriasiform phenotype including skin, nail and joint changes. (FIG. 3A,3B) Lesional Rac1 skin demonstrated psoriasiform hyperplasia, hypogranulosis, a mixed inflammatory infiltrate, (FIG. 3C, arrows) dilated vessels in dermal papillae and (FIG. 3D) foci of marked parakeratosis. (FIG. 3E,3F) Wound induced psoriasiform hyperplasia (Koebner phenomenon). (FIG. 3G, 3H, 3I,3J-lower arrows) Mucosa (but not perimucosal skin) spared from proliferative/inflammatory changes. (FIG. 3K, arrow) Rac1 mice with joint involvement showed neutrophillic infiltrates near joint spaces (FIG. 3L). (FIG. 3M,3N, arrow) Nail changes ranged from ridging to marked thickening/onycholysis and nail matrix showed psoriasiform hyperplasia. (FIG. 30) FACS analysis showed increased CD4+ and CD8+ lymphocytes, neutrophils and DCs in Rac1 lesional skin. (FIG. 3P) Increased CD3+ and (FIG. 3Q) RORγ+ lymphocytes and (FIG. 3R) CD68+ cells (Munro's microabscesses) in the stratum corneum. (FIG. 3S) NOD SCID Rac1 backcrossing resulted in a marked reduction in epidermal thickness and suprabasal proliferation (Ki67) despite persistent Rac1 (but not RhoA) activity, and absence of tail and limb joint abnormalities. CD3/RORy/CD68/Ki67/Rac1GTP/RhoAGTP/PSTAT3-red, DNA-blue. Scalebar 100/200 μm.

FIG. 4A-4H: Transcriptional signature of Rac1 activity in human psoriasis skin. (FIG. 4A) Differentially expressed genes (DEGs) in Rac1 (n=3) compared littermate control skin (n=3), 46% (284) upregulated and 54% (333) repressed. (FIG. 4B) Biological functions (−log P upper axis-grey, n-genes lower axis-red); (FIG. 4C) Canonical pathways (−log P upper axis-grey, ratio lower axis-red); and (FIG. 4D) transcription factors and cytokines (−log P upper axis-grey, activation z-score lower axis-red) of the DEG signature in Rac1-skin. (FIG. 4E) Overlap (p<0.05) between orthologous DEGs in Rac1 and human psoriatic skin. (FIG. 4F) Overlapping DEGs included KRT16, S100A9, OAS1, PTGES, IL36A/RN, STAT3 and CGNL1. (FIG. 4G) Overlap enriched for psoriasis-associated canonical pathways and (FIG. 4H) biological functions. (A,F,G ANOVA, F Hypergeometric mean, B-E, H-J Fisher Exact test).

FIG. 5A-5M: V12Rac1 mice exhibit differential regulation of epidermal proteins and transcription factors involved in human psoriasis. Expression of (FIG. 5A) TGFα, (FIG. 5B) CD11c (upper)/1123p19 (lower) and (FIG. 5C) CARD14 in V12Rac1-, WT-, psoriasis lesional and control skin (n=6). (FIG. 5D-5G) Expression of epidermal PSTAT3/P-p65 and COL7/DSG3 with representative z stacks (z) of V12Rac1 and psoriasis. TGFα/IL23p19/COL7/DSG3-green, CD11c/CARD14/PSTAT3/PRELA—red, DNA—blue. Scalebars 50/10 (z stacks) μm. (FIG. 5H) Western blot and (FIG. 5I,5J) quantification of PSTAT3 and acetyl p65 relative actin from V12Rac1- and WT skin (n=3 each). (FIG. 5K) Reduced PSTAT3 in NOD-SCID Rac1 mice. (FIG. 5L) RT-qPCR of mRNA from wholeskin (white-) or epidermis (grey bars) from 1 week old V12Rac1 or WT mice. (FIG. 5M) Inflammatory markers in 3 week old V12Rac1- or WT mouse serum (n=3 per condition). *=P<0.05, **=P<0.005, ***=<P<0.0005. (I,J: unpaired t-test, L: Mann-Whitney ranked test, M:Tukey's multiple comparison test). Error bars SEM. PSO: human psoriasis. CTL: human control. Rac1: V12 Rac1 mouse. WT: Wild-type mouse.

FIG. 6A-6P: Rac1-dependent signaling in psoriatic keratinocytes affects psoriasis-associated cytokine production, and drives hyper-proliferation and hypo-differentiation through ZNF750. Expression of (FIG. 6A) CARD14, (FIG. 6B) IFIH1 and (FIG. 6C) PSTAT3 by IDIF in LacZPHKC, N17Rac1PHKC, V12NHKC or LacZNHKC. Scalebars 100/50/25 μm. z=z stacks. CARD14/IFIH1/PSTAT3-red, Rac1GTP-green, DNA-blue. (FIG. 6D,6E) Quantification of CARD14 and IFIH1 intracellular expression. (FIG. 6F) Western blots and (FIG. 6G) quantification of PSTAT3 after 0 and 24 h 1117A/F stimulation. (n=3 per condition). (FIG. 6H, 6J) Western blots and (FIG. 6I, 6K) quantification of PSTAT3 after 0 10 and 90 minutes stimulation with EGF or TNFα. (FIG. 6L) RT-qPCR of mRNA from undifferentiated or (FIG. 6M) differentiated LacZ or V12Rac1NHKC after ZNF750 or pLEX control (CTL) overexpression. (FIG. 6N) V12Rac1 protein expression (+), with and without ectopic ZNF750 (+) in undifferentiated (left) or differentiated (right). Ratios of ZNF750 V12 to LacZ 0.42, and 0.82 with ectopic ZNF750. (FIG. 6O) MTT assay of V12Rac1NHKC and LacZNHKC with ZNF750 or CTL. (FIG. 6P) Rac1GTP pulldown and quantification of siRNA ZNF750 PHKCs compared to scramble siRNA (SCR). *P=<0.05, **=P<0.05. Error bars SEM. (G,I,K,P Unpaired t-test, D,E,L,M,O Mann-Whitney ranked test). G,L,M,P n=3,I,K,N n=2 replicates per condition. PHKC/PSO: primary human psoriatic keratinocyte. NHKC/CTL: primary normal human keratinocytes. Rac1KC/V12: V12Rac1 NHKC. N17Rac1/N17: N17 PHKC.

FIG. 7A-7D: Epidermal Rac1 promotes an immunoproliferative psoriasis phenotype. (FIG. 7A,7B) PSOKC or NHKC cultured atop devitalized dermis and xenografted to NOD/SCID mice or after intradermal injection of autologous PBMCs and retroviral keratinocyte-specific transduction of N17 or LacZ control. DSG3-red, Rac1GTP-green, DNA-blue. Scalebars 100/25 μm. (FIG. 7C) Luminex panel of cytokine expression in supernatants of LacZPHKC, N17PHKC, LacZNHKC or V12NHKC alone or in co-cultures with PBMCs. Relative expression values normalized to only PBMC (dotted line). All conditions were in duplicates. Error bars SEM. (FIG. 7D) Schematic model of the potential effect of epidermal Rac1 activation in psoriasis development. KC: keratinocyte. PBMC: peripheral blood mononuclear cells. PSOKC/PSO: Psoriatic primary human keratinocyte. N17PHKC/N17: N17 PHKC. NHKC/CTL: Control primary human keratinocyte.

### DETAILED DESCRIPTION OF THE EMBODIMENTS

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. In this specification and the appended claims, the singular forms “a,” “an” and “the” include plural reference unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, illustrative methods, devices and materials are now described.

All publications mentioned herein are incorporated herein by reference for the purpose of describing and disclosing the subject components of the invention that are described in the publications, which components might be used in connection with the presently described invention.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

Chronic Plaque Psoriasis. Chronic plaque psoriasis (also referred to as psoriasis vulgaris) is the most common form of psoriasis. Chronic plaque psoriasis is characterized by raised reddened patches of skin, ranging from coin-sized to much larger. In chronic plaque psoriasis, the plaques may be single or multiple, they may vary in size from a few millimeters to several centimeters. The plaques are usually red with a scaly surface, and reflect light when gently scratched, creating a “silvery” effect. Lesions (which are often symmetrical) from chronic plaque psoriasis occur all over body, but with predilection for extensor surfaces, including the knees, elbows, lumbosacral regions, scalp, and nails. Occasionally chronic plaque psoriasis can occur on the penis, vulva and flexures, but scaling is usually absent. Diagnosis of patients with chronic plaque psoriasis is usually based on the clinical features described above. In particular, the distribution, color and typical silvery scaling of the lesion in chronic plaque psoriasis are characteristic of chronic plaque psoriasis.

Guttate Psoriasis. Guttate psoriasis refers to a form of psoriasis with characteristic water drop shaped scaly plaques. Flares of guttate psoriasis generally follow an infection, most notably a streptococcal throat infection. Diagnosis of guttate psoriasis is usually based on the appearance of the skin, and the fact that there is often a history of recent sore throat.

Inverse Psoriasis. Inverse psoriasis is a form of psoriasis in which the patient has smooth, usually moist areas of skin that are red and inflamed, which is unlike the scaling associated with plaque psoriasis. Inverse psoriasis is also referred to as intertiginous psoriasis or flexural psoriasis. Inverse psoriasis occurs mostly in the armpits, groin, under the breasts and in other skin folds around the genitals and buttocks, and, as a result of the locations of presentation, rubbing and sweating can irritate the affected areas.

Pustular Psoriasis. Pustular psoriasis, also referred to as palmar plantar psoriasis, is a form of psoriasis that causes pus-filled blisters that vary in size and location, but often occur on the hands and feet. The blisters may be localized, or spread over large areas of the body. Pustular psoriasis can be both tender and painful, can cause fevers.

Erythrodermic Psoriasis. Erythrodermic psoriasis is a particularly inflammatory form of psoriasis that often affects most of the body surface. It may occur in association with von Zumbusch pustular psoriasis. It is a rare type of psoriasis, occurring once or more during the lifetime of 3 percent of people who have psoriasis. It generally appears on people who have unstable plaque psoriasis. Widespread, fiery redness and exfoliation of the skin characterize this form. Severe itching and pain often accompanies it. Erythrodermic psoriasis causes protein and fluid loss that can lead to severe illness. Edema (swelling from fluid retention), especially around the ankles, may develop, along with infection. Erythrodermic psoriasis also can bring on pneumonia and congestive heart failure. People with severe cases often require hospitalization. Erythrodermic psoriasis can occur abruptly at the first signs of psoriasis or it can come on gradually in people with plaque psoriasis. Combination treatments are frequently required, for example topical products and one or two systemic medications.

The term “sensitivity” and “sensitive” when made in reference to treatment is a relative term which refers to the degree of effectiveness of a treatment compound in lessening or decreasing the symptoms of the disease being treated. For example, the term “increased sensitivity” when used in reference to treatment of a cell or patient refers to an increase of, at least a 5%, or more, in the effectiveness in lessening or decreasing the symptoms of psoriasis when measured using any methods well-accepted in the art.

As used herein, and unless otherwise specified, the term “therapeutically effective amount” of a compound is an amount sufficient to provide a therapeutic benefit in the treatment or management of psoriasis, or to delay or minimize one or more symptoms associated with psoriasis. A therapeutically effective amount of a compound means an amount of therapeutic agent, alone or in combination with other therapies, which provides a therapeutic benefit in the treatment or management of psoriasis. The term “therapeutically effective amount” can encompass an amount that improves overall therapy, reduces or avoids symptoms or causes of psoriasis, or enhances the therapeutic efficacy of another therapeutic agent.

The term “likelihood” generally refers to an increase in the probability of an event. The term “likelihood” when used in reference to the effectiveness of a patient response generally contemplates an increased probability that the symptoms of psoriasis will be lessened or decreased.

The terms “determining”, “measuring”, “evaluating”, “assessing” and “assaying” as used herein generally refer to any form of measurement, and include determining if an element is present or not. These terms include both quantitative and/or qualitative determinations. Assessing may be relative or absolute. “Assessing the presence of” can include determining the amount of something present, as well as determining whether it is present or absent.

The term “sample” as used herein relates to a material or mixture of materials, typically, although not necessarily, in fluid form, containing one or more components of interest.

“Biological sample” as used herein refers to a sample obtained from a biological subject, including sample of biological tissue or fluid origin, obtained, reached, or collected in vivo or in situ. A biological sample also includes samples from a region of a biological subject containing precancerous or cancer cells or tissues. Such samples can be, but are not limited to, organs, tissues, fractions and cells isolated from a mammal. Exemplary biological samples include but are not limited to cell lysate, a cell culture, a cell line, a tissue, oral tissue, gastrointestinal tissue, an organ, an organelle, a biological fluid, a blood sample, a urine sample, a skin sample, and the like. Preferred biological samples include but are not limited to whole blood, partially purified blood. PBMCs, tissue biopsies, and the like.

The term “combination” as in the phrase “a first agent in combination with a second agent” includes co-administration of a first agent and a second agent, which for example may be dissolved or intermixed in the same pharmaceutically acceptable carrier, or administration of a first agent, followed by the second agent, or administration of the second agent, followed by the first agent. The present invention, therefore, includes methods of combination therapeutic treatment and combination pharmaceutical compositions.

The term “concomitant” as in the phrase “concomitant therapeutic treatment” includes administering an agent in the presence of a second agent. A concomitant therapeutic treatment method includes methods in which the first, second, third, or additional agents are co-administered. A concomitant therapeutic treatment method also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and a second actor may to administer to the subject a second agent, and the administering steps may be executed at the same time, or nearly the same time, or at distant times, so long as the first agent (and additional agents) are after administration in the presence of the second agent (and additional agents). The actor and the subject may be the same entity (e.g., human).

As used herein, the term “dose amount” refers to the quantity, e.g., milligrams (mg), of the substance which is administered to the subject. In one embodiment, the dose amount is a fixed dose, e.g., is not dependent on the weight of the subject to which the substance is administered. In another embodiment, the dose amount is not a fixed dose, e.g., is dependent on the weight of the subject to which the substance is administered, or for a topical therapy a dose may be related to the surface area that is treated, e.g. dose/m² of skin.

Exemplary dose amounts, e.g., fixed dose amounts, for use treating an adult human by the methods of the invention include, about 0.01 mg, about 0.05 mg, about 0.1 mg, about 0.5 mg, about 1 mg, about 5 mg, about 10 mg, about 50 mg, about 100 mg, about 500 mg, or more.

Exemplary dose amounts, e.g., dose amounts for topical use treating an adult human by the methods of the invention include, about 0.01 mg/m² surface area, about 0.05 mg/m² surface area, about 0.1 mg/m² surface area, about 0.5 mg/m² surface area, about 1 mg/m² surface area, about 5 mg/m² surface area, about 10 mg/m² surface area, about 50 mg/m² surface area, about 100 mg/m² surface area, about 500 mg/m² surface area, or more.

Ranges intermediate to the above-recited ranges are also contemplated by the invention. For example, ranges having any one of these values as the upper or lower limits are also intended to be part of the invention, e.g., about 0.01 mg to about 100 mg, about 1 mg to about 10 mg, etc.

As used herein, the term “periodicity” as it relates to the administration of a substance refers to a (regular) recurring cycle of administering the substance to a subject. In one embodiment, the recurring cycle of administration of the substance to the subject achieves a therapeutic objective. The periodicity of administration of the substance may be about once a week, once every other week, about once every three weeks, about once every 4 weeks, about once every 5 weeks, about once every 6 weeks, about once every 7 weeks, about once every 8 weeks, about once every 9 weeks, about once every 10 weeks, about once every 11 weeks, about once every 12 weeks, about once every 13 weeks, about once every 14 weeks, about once every 15 weeks, about once every 16 weeks, about once every 17 weeks, about once every 18 weeks, about once every 19 weeks, about once every 20 weeks, about once every 21 weeks, about once every 22 weeks, about once every 23 weeks, about once every 24 weeks, about once every 5-10 days, about once every 10-20 days, about once every 10-50 days, about once every 10-100 days, about once every 10-200 days, about once every 25-35 days, about once every 20-50 days, about once every 20-100 days, about once every 20-200 days, about once every 30-50 days, about once every 30-90 days, about once every 30-100 days, about once every 30-200 days, about once every 50-150 days, about once every 50-200 days, about once every 60-180 days, or about once every 80-100 days. Periodicities intermediate to the above-recited times are also contemplated by the invention. Ranges intermediate to the above-recited ranges are also contemplated by the invention. For example, ranges having any one of these values as the upper or lower limits are also intended to be part of the invention, e.g., about 110 days to about 170 days, about 160 days to about 220 days, etc.

The “duration of a periodicity” refers to a time over which the recurring cycle of administration occurs. For example, a duration of the periodicity of administration of a substance may be may be up to about 4 weeks, up to about 8 weeks, up to about 12 weeks, up to about 16 weeks or more, up to about 20 weeks, up to about 24 weeks, up to about 28 week, up to about 32 weeks or more, during which the periodicity of administration is about once every week. For example, a duration of the periodicity may be about 6 weeks during which the periodicity of administration is about once every 4 weeks, e.g., the substance is administered at week zero and at week four.

In one embodiment, the duration of periodicity is for a length of time necessary or required to achieve a therapeutic objective, e.g., treatment, maintenance of treatment, etc. e.g., maintain a PASI 50, PASI 75, PASI 90, PASI 100 score or PGA of 0 or 1 score. Durations of a periodicity intermediate to the above-recited times are also contemplated by the invention.

As used herein, and unless otherwise specified, the terms “treat,” “treating” and “treatment” refer to an action that occurs while a patient is suffering from psoriasis, which reduces the severity of psoriasis, or retards or slows the progression of the psoriasis, or achieving or maintaining a therapeutic objective. An “effective patient response” refers to any increase in the therapeutic benefit to the patient. An “effective patient psoriasis response” can be, for example, a 5%, 10%, 25%, 50%, or 100% decrease in the physical symptoms of psoriasis.

“Treatment of or “treating” psoriasis may mean achieving or maintaining a PGA score of 0/1 or a PASI 50, PASI 75, PASI 90, or PASI 100 response score for a period of time during or following treatment (e.g., for at least 2, 4, 6, 8, 10, 12, 14, 16, 18, 20, 22, 24, 26, 28, 30, 32, 34, 36, 38, 40, 42, 46, 48, 50, 52, 54, 56, 58 or 60 weeks or longer). “Treatment of or “treating” psoriasis may also mean achieving or maintaining a health-related quality of life (HRQOL) outcome. HRQOL outcomes include Dermatology Life Quality Index (DLQI), visual analog scales for Ps-related (VAS-Ps) and psoriatic arthritis-related (VAS-PsA) pain, Short Form 36 Health Survey Mental (MCS) and Physical (PCS) Component Summary scores, and Total Activity Impairment (TAI) scores.

“Treatment of or “treating” psoriasis may also mean achieving or maintaining a minimum clinically important difference (MCID) for any of the HRQOL outcomes provided herein, e.g., any one or combination of DLQI, VAS-Ps, VAS-PsA, MCS, PCS and TAI.

“Treatment of” or “treating” psoriasis may also mean achieving or maintaining a minimum clinically important difference (MCID) response rate for any of the HRQOL outcomes provided herein, e.g., any one or combination of DLQI, VAS-Ps, VAS-PsA, MCS, PCS and TAI. “Treatment of or “treating” psoriasis may also mean achieving or maintaining a clinically meaningful reduction in any of the HRQOL outcomes provided herein, e.g., any one or combination of DLQI, VAS-Ps, VAS-PsA, MCS, PCS and TAI.

“Treatment of or “treating” psoriasis may also mean achieving or maintaining a Nail Psoriasis Severity Index (NAPSI) score for a period of time during or following treatment (e.g., for at least 2, 4, 6, 8, 10, 12, 14, 16, 18, 20, 22, 24, 26, 28, 30, 32, 34, 36, 38, 40, 42, 46, 48, 50, 52, 54, 56, 58 or 60 weeks or longer).

“Treatment of” or “treating” psoriasis may also mean achieving or maintaining any of the outcomes provided herein in a certain percentage of a population of subjects (e.g., in at least about 10%, 15%, 20%, 25%, 30%, 35%, 40%, 45%, 50%, 55%, 60%, 65%, 70%, 75%, 80%, 85%, 90%, 95%, 99%, or 100% of a population of subjects).

The term “kit” as used herein refers to a packaged product comprising components with which to administer the epithelial ion channel blocker of the invention for treatment of psoriasis. The kit preferably comprises a box or container that holds the components of the kit. The box or container may be affixed with a label or a Food and Drug Administration approved protocol. The box or container holds components of the invention which are preferably contained within plastic, polyethylene, polypropylene, ethylene, or propylene vessels. The vessels can be capped-tubes or bottles. The kit can also include instructions for use.

Transgenic Animals. The term “transgene” is used herein to describe genetic material that has been or is about to be artificially inserted into the genome of a mammalian cell, particularly a mammalian cell of a living animal. The transgene is used to transform a cell, meaning that a permanent or transient genetic change, preferably a permanent genetic change, is induced in a cell following incorporation of exogenous DNA. A permanent genetic change is generally achieved by introduction of the DNA into the genome of the cell. Vectors for stable integration include plasmids, retroviruses and other animal viruses, YACs, and the like.

Transgenic animals comprise an exogenous nucleic acid sequence present as an extrachromosomal element or stably integrated in all or a portion of its cells, especially in germ cells. Unless otherwise indicated, it will be assumed that a transgenic animal comprises stable changes to the germline sequence. During the initial construction of the animal, “chimeras” or “chimeric animals” are generated, in which only a subset of cells have the altered genome. Chimeras are primarily used for breeding purposes in order to generate the desired transgenic animal. Animals having a heterozygous alteration are generated by breeding of chimeras. Male and female heterozygotes are typically bred to generate homozygous animals.

Transgenic animals fall into two groups, colloquially termed “knockouts” and “knockins”. In the present invention, knockin animals comprise an activated Rac1 gene, including without limitation the V12 sequence as disclosed in the Examples. The gene is operably linked to a promoter active in epidermal tissue, preferable selectively active in epidermal tissue. Keratin promoters, e.g. Keratin 14 promoter, are conveniently used.

The exogenous gene may be from a different species than the animal host, or is otherwise altered in its coding or non-coding sequence. The introduced gene may be a wild-type gene, naturally occurring polymorphism, or a genetically manipulated sequence, for example having deletions, substitutions or insertions in the coding or non-coding regions. By “operably linked” is meant that a DNA sequence and a regulatory sequence(s) are connected in such a way as to permit gene expression when the appropriate molecules, e.g. transcriptional activator proteins, are bound to the regulatory sequence(s).

Transgenic mice may be generated by injection of the DNA construct into the pronucleus of fertilized oocytes.

The transgenic animals and xenografted animals may be used in a wide variety of ways, e.g. in gene discovery; for dissection of Rac signaling pathways; for screening assays; and the like.

The animals and cells derived therefrom may be used for screening candidate therapies modifiers, i.e. compounds and factors that affect Rac1 signaling pathways and psoriasis. A wide variety of assays may be used for this purpose, including immunoassays for protein binding; determination of cell growth, differentiation and functional activity; production of hormones; psoriasis phenypes of the skin, and the like.

Typically the candidate compound will be added to the cells and/or animal including topical administration, and the response of the cells monitored through evaluation of cell surface phenotype, functional activity, patterns of gene expression, and the like. Through use of the subject transgenic animals or cells derived therefrom, one can identify ligands or substrates that treat psoriasis through targeting activated Rac1. Depending on the particular assay, whole animals may be used, or cell derived therefrom. Cells may be freshly isolated from an animal, or may be immortalized in culture.

The term “agent” as used herein describes any molecule, e.g. protein or pharmaceutical, with the capability of affecting the biological action of Rac1. Generally a plurality of assay mixtures are run in parallel with different agent concentrations to obtain a differential response to the various concentrations. Typically, one of these concentrations serves as a negative control, i.e. at zero concentration or below the level of detection. Screening may be directed to known pharmacologically active compounds and chemical analogs thereof.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components is added in any order that provides for the requisite binding. Incubations are performed at any suitable temperature, typically between 4 and 40.degree. C. Incubation periods are selected for optimum activity, but may also be optimized to facilitate rapid high-throughput screening. Typically between 0.1 and 1 hours will be sufficient.

For example, detection may utilize staining of cells or histological sections, performed in accordance with conventional methods. The antibodies of interest are added to the cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. The absence or presence of antibody binding may be determined by various methods, including flow cytometry of dissociated cells, microscopy, radiography, scintillation counting, etc.

Gene expression in the cells of the invention may be assessed following a candidate treatment or experimental manipulation. The expressed set of genes may be compared with a variety of cells of interest, e.g. keratinocytes, etc., as known in the art. Any suitable qualitative or quantitative methods known in the art for detecting specific mRNAs can be used. mRNA can be detected by, for example, hybridization to a microarray, in situ hybridization in tissue sections, by reverse transcriptase-PCR, or in Northern blots containing poly A+ mRNA. One of skill in the art can readily use these methods to determine differences in the size or amount of mRNA transcripts between two samples. For example, the level of particular mRNAs in mast cells is compared with the expression of the mRNAs in a reference sample.

In another screening method, the test sample is assayed at the protein level. Methods of analysis may include 2-dimensional gels; mass spectroscopy; analysis of specific cell fraction, e.g. lysosomes; and other proteomics approaches. For example, detection can utilize staining of cells or histological sections (e.g., from a biopsy sample) with labeled antibodies, performed in accordance with conventional methods. Cells can be permeabilized to stain cytoplasmic molecules. In general, antibodies that specifically bind a differentially expressed polypeptide of the invention are added to a sample, and incubated for a period of time sufficient to allow binding to the epitope, usually at least about 10 minutes. The antibody can be detectably labeled for direct detection (e.g., using radioisotopes, enzymes, fluorescers, chemiluminescers, and the like), or can be used in conjunction with a second stage antibody or reagent to detect binding (e.g., biotin with horseradish peroxidase-conjugated avidin, a secondary antibody conjugated to a fluorescent compound, e.g. fluorescein, rhodamine, Texas red, etc.). The absence or presence of antibody binding can be determined by various methods, including flow cytometry of dissociated cells, microscopy, radiography, scintillation counting, etc. Any suitable alternative methods can of qualitative or quantitative detection of levels or amounts of differentially expressed polypeptide can be used, for example ELISA, western blot, immunoprecipitation, radioimmunoassay, etc.

Where an animal is being tested, the severity of skin inflammation may be assessed. For example, the severity of psoriasis is measured by the Psoriasis Area Severity Index (PASI) (see e.g., Fleischer et al. (1999), J. Dermatol. 26:210-215 and Tanew et al. (1999), Arch Dermatol. 135:519-524) or various psoriasis global assessment scores such as Physician's Global Assessment (PGA) which are well-known to those skilled in the art of clinical trials for psoriasis. Typically, in a clinical trial (e.g., a phase II or phase III trial), the improvement in PASI or score in the patients treated with a candidate agent, relative to the control group of patients receiving no treatment or placebo or another agent, will be statistically significant, for example at the p=0.05 or 0.01 or even 0.001 level.

Formulations. The present invention provides pharmaceutically acceptable compositions which comprise a therapeutically-effective amount of at least one Rac1 inhibitor, particularly an inhibitor identified by the screening methods disclosed herein, and optionally combined with one or more additional agents for treatment of psoriasis, formulated together with one or more pharmaceutically acceptable excipients. The active ingredients and excipient(s) may be formulated into compositions and dosage forms according to methods known in the art. As described in detail below, the pharmaceutical compositions of the present invention may be specially formulated for administration in solid or liquid form, including those adapted for the following: oral administration, for example, tablets, capsules, powders, granules, pastes for application to the tongue, aqueous or non-aqueous solutions or suspensions, drenches, or syrups; parenteral administration, for example, by subcutaneous, intramuscular or intravenous injection as, for example, a sterile solution or suspension. In other embodiments the formulation is provided for topical application, for example, as a lotion, cream, ointment, spray, patch, microneedle array, etc. applied to the skin.

The phrase “pharmaceutically acceptable” is employed herein to refer to those compounds, materials, compositions, and/or dosage forms which are, within the scope of sound medical judgment, suitable for use in contact with the tissues of the subject with toxicity, irritation, allergic response, or other problems or complications, commensurate with a reasonable benefit/risk ratio.

The phrase “pharmaceutically-acceptable excipient” as used herein refers to a pharmaceutically-acceptable material, composition or vehicle, such as a liquid or solid filler, diluent, carrier, manufacturing aid (e.g., lubricant, talc magnesium, calcium or zinc stearate, or steric acid), solvent or encapsulating material, involved in carrying or transporting the therapeutic compound for administration to the subject. Each excipient should be “acceptable” in the sense of being compatible with the other ingredients of the formulation and not injurious to the subject. Some examples of materials which can serve as pharmaceutically-acceptable excipients include: ethanol, sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations. If desired, certain sweetening and/or flavoring and/or coloring agents may be added. Other suitable excipients can be found in standard pharmaceutical texts, e.g. in “Remington's Pharmaceutical Sciences”, The Science and Practice of Pharmacy, 19.sup.th Ed. Mack Publishing Company, Easton, Pa., (1995).

Excipients are added to the composition for a variety of purposes. Diluents increase the bulk of a solid pharmaceutical composition, and may make a pharmaceutical dosage form containing the composition easier for the patient and caregiver to handle. Diluents for solid compositions include, for example, microcrystalline cellulose, microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose, hydroxypropyl methyl cellulose, liquid glucose, magnesium aluminum silicate, maltodextrin, methylcellulose, polymethacrylates, povidone, pregelatinized starch, sodium alginate and starch. The dissolution rate of a compacted solid pharmaceutical composition in the subjects's stomach may be increased by the addition of a disintegrant to the composition. Disintegrants include alginic acid, carboxymethylcellulose calcium, carboxymethylcellulose sodium, colloidal silicon dioxide, croscarmellose sodium, crospovidone, guar gum, magnesium aluminum silicate, methyl cellulose, microcrystalline cellulose, polacrilin potassium, powdered cellulose, pregelatinized starch, sodium alginate, sodium starch glycolate and starch.

In liquid pharmaceutical compositions of the present invention, the agent and any other solid excipients are dissolved or suspended in a liquid carrier such as water, water-for-injection, vegetable oil, alcohol, polyethylene glycol, propylene glycol or glycerin. Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol. Liquid pharmaceutical compositions of the present invention may also contain a viscosity enhancing agent to improve the mouth-feel of the product and/or coat the lining of the gastrointestinal tract. Sweetening agents such as sorbitol, saccharin, sodium saccharin, sucrose, aspartame, fructose, mannitol and invert sugar may be added to improve the taste. Flavoring agents and flavor enhancers may make the dosage form more palatable to the patient. Preservatives and chelating agents such as alcohol, sodium benzoate, butylated hydroxy toluene, butylated hydroxyanisole and ethylenediamine tetraacetic acid may be added at levels safe for ingestion to improve storage stability. Selection of excipients and the amounts used may be readily determined by the formulation scientist based upon experience and consideration of standard procedures and reference works in the field.

In several embodiments of the invention the Rac1 inhibitor isformulated for topical application to the skin Various specific formulations are provided, including lotions, gels, liquids, patches, intralesional injection, and the like. A typical dose for a topical formulation in lotion or liquid form is from about 1 μl to about 100 μl to about 1 ml, to about 10 ml, applied in a lotion, cream, gel, etc. to the affected skin.

In general, the subject formulations will typically contain at least about 1 μg/ml active agent, at least about 10 μg/ml, at least about 50 μg/ml, at least about 100 μg/ml, at least about 500 μg/ml, and not more than about 100 mg/ml. In some embodiments the formulation comprises at least about 0.1 mM, at least about 0.05, at least about 1 mM, at least about 5 mM, at least about 10 mM, at least about 50 mM. The active agents of the present invention are formulated at an effective concentration within the subject formulations, meaning at a concentration that provides the intended benefit when applied topically.

The dose of active agent is as described above with respect to the surface area to be treated, where the dose may be up to about 0.01 mg/kg body weight, up to about 0.05 mg/kg body weight, up to about 0.1 mg/kg body weight, up to about 0.5 mg/kg body weight, up to about 1 mg/kg body weight, up to about 2 mg/kg body weight, up to about 5 mg/kg body weight, up to about 10 mg/kg body weight.

Administration may be every 6 hours, every 12 hours, every 24 hours, every 48 hours, every 3 days, every 4 days, every 5 days, weekly, biweekly, monthly, etc. In various of these embodiments, the therapeutically effective dose is administered on consecutive days for at least a week, at least a month, at least a year, or on as needed basis for the rest of the patient's life. The therapeutically effective dose, e.g. of Benzamil, or pharmaceutically acceptable salt thereof, can be about 10-500 mg/day, about 50-400 mg/day, about 100-200 mg/day, or about 120-180 mg/day. Benzamil or pharmaceutically acceptable salt thereof, can be administered to a subject at about 1-110 mg daily, 1-100 mg twice a day, 1-100 mg. every other day, as needed.

Examples are provided herein of dosages useful for treatment of an animal model. As is known in the art, in order to convert dosage from, for example, a mouse to a human, the animal dose should not be extrapolated to a human equivalent dose (HED) by a simple conversion based on body weight. The more appropriate conversion of drug doses from animal studies to human studies, uses the body surface area (BSA) normalization method. BSA correlates well across several mammalian species with several parameters of biology, including oxygen utilization, caloric expenditure, basal metabolism, blood volume, circulating plasma proteins, and renal function. See, for example, Reagan-Shaw et al. (2008) The FASEB Journal 22(3), 659-661, herein specifically incorporated by reference. The appropriate dose for a human may be roughly 1/10^{th} to 1/20^{th} of the dose for a mouse. See also, FDA guidance for Estimating the Maximum Safe Starting Dose in Initial Clinical Trials for Therapeutics in Adult Healthy Volunteers.

In some embodiments, the topical formulation comprises skin penetration enhancers. Such enhancers reversibly decrease skin barrier resistance, and include without limitation, sulphoxides (such as dimethylsulphoxide, DMSO), azones (e.g. laurocapram), pyrrolidones (for example 2-pyrrolidone, 2P), alcohols and alkanols (ethanol, or decanol), glycols (for example propylene glycol, PG, a common excipient in topically applied dosage forms), surfactants (also common in dosage forms) and terpenes.

Topical formulations include lotions, gels, creams, etc. Such formulations may include a pharmaceutically acceptable vehicle to act as a dilutant, dispersant or carrier for the active agent(s), so as to facilitate distribution when the composition is applied to the skin. Vehicles other than or in addition to water can include liquid or solid emollients, solvents, humectants, thickeners and powders. The vehicle will usually form from 5% to 99.9%, preferably from 25% to 80% by weight of the composition, and can, in the absence of other cosmetic adjuncts, form the balance of the composition. The compositions may be in the form of aqueous, aqueous/alcoholic or oily solutions; dispersions of the lotion or serum type; anhydrous or lipophilic gels; emulsions of liquid or semi-liquid consistency, which are obtained by dispersion of a fatty phase in an aqueous phase (0/W) or conversely (W/O); or suspensions or emulsions of smooth, semi-solid or solid consistency of the cream or gel type. These compositions are formulated according to the usual techniques as are well known to this art.

When formulated as an emulsion, the proportion of the fatty phase may range from 5% to 80% by weight, and preferably from 5% to 50% by weight, relative to the total weight of the composition. Oils, emulsifiers and co-emulsifiers incorporated in the composition in emulsion form are selected from among those used conventionally in the cosmetic or dermatological field. The emulsifer and coemulsifier may be present in the composition at a proportion ranging from 0.3% to 30% by weight, and preferably from 0.5% to 20% by weight, relative to the total weight of the composition. When the lotions are formulated as an oily solution or gel, the fatty phase may constitute more than 90% of the total weight of the composition.

Formulations may also contain additives and adjuvants which are conventional in the cosmetic, pharmaceutical or dermatological field, such as hydrophilic or lipophilic gelling agents, hydrophilic or lipophilic active agents, preservatives, antioxidants, solvents, fragrances, fillers, bactericides, odor absorbers and dyestuffs or colorants. The amounts of these various additives and adjuvants are those conventionally used in the field, and, for example, range from 0.01% to 10% of the total weight of the composition. Depending on their nature, these additives and adjuvants may be introduced into the fatty phase, into the aqueous phase.

Exemplary oils which may be used according to this invention include mineral oils (liquid petrolatum) and solid oils, e.g. petrolatum, plant oils (liquid fraction of karite butter, sunflower oil), animal oils (perhydrosqualen(e), synthetic oils (purcellin oil), silicone oils (cyclomethicone) and fluoro oils (perfluoropolyethers). Fatty alcohols, fatty acids (stearic acid) and waxes (paraffin wax, carnauba wax and beeswax) may also be used as fats. Emulsifiers which may be used include glyceryl stearate, polysorbate 60, PEG-6/PEG-32/glycol stearate mixture, etc. Solvents which may be used include the lower alcohols, in particular ethanol and isopropanol, and propylene glycol. Hydrophilic gelling agents include carboxyvinyl polymers (carbomer), acrylic copolymers such as acrylate/alkylacrylate copolymers, polyacrylamides, polysaccharides, such as hydroxypropylcellulose, natural gums and clays, and, as lipophilic gelling agents, representative are the modified clays such as bentones, fatty acid metal salts such as aluminum stearates and hydrophobic silica, or ethylcellulose and polyethylene.

An oil or oily material may be present, together with an emollient to provide either a water-in-oil emulsion or an oil-in-water emulsion, depending largely on the average hydrophilic-lipophilic balance (HLB) of the emollient employed. Levels of such emollients may range from about 0.5% to about 50%, preferably between about 5% and 30% by weight of the total composition. Emollients may be classified under such general chemical categories as esters, fatty acids and alcohols, polyols and hydrocarbons. Esters may be mono- or di-esters. Acceptable examples of fatty di-esters include dibutyl adipate, diethyl sebacate, diisopropyl dimerate, and dioctyl succinate. Acceptable branched chain fatty esters include 2-ethyl-hexyl myristate, isopropyl stearate and isostearyl palmitate. Acceptable tribasic acid esters include triisopropyl trilinoleate and trilauryl citrate. Acceptable straight chain fatty esters include lauryl palmitate, myristyl lactate, oleyl eurcate and stearyl oleate. Preferred esters include coco-caprylate/caprate (a blend of coco-caprylate and coco-caprate), propylene glycol myristyl ether acetate, diisopropyl adipate and cetyl octanoate.

Suitable fatty alcohols and acids include those compounds having from 10 to 20 carbon atoms. Especially preferred are such compounds such as cetyl, myristyl, palmitic and stearyl alcohols and acids. Among the polyols which may serve as emollients are linear and branched chain alkyl polyhydroxyl compounds. For example, propylene glycol, sorbitol and glycerin are preferred. Also useful may be polymeric polyols such as polypropylene glycol and polyethylene glycol. Butylene and propylene glycol are also especially preferred as penetration enhancers.

Exemplary hydrocarbons which may serve as emollients are those having hydrocarbon chains anywhere from 12 to 30 carbon atoms. Specific examples include mineral oil, petroleum jelly, squalene and isoparaffins.

Another category of functional ingredients for lotions are thickeners. A thickener will usually be present in amounts anywhere from 0.1 to 20% by weight, preferably from about 0.5% to 10% by weight of the composition. Exemplary thickeners are cross-linked polyacrylate materials available under the trademark Carbopol. Gums may be employed such as xanthan, carrageenan, gelatin, karaya, pectin and locust beans gum. Under certain circumstances the thickening function may be accomplished by a material also serving as a silicone or emollient. For instance, silicone gums in excess of 10 centistokes and esters such as glycerol stearate have dual functionality. Powders may be incorporated into a lotion. These powders include chalk, talc, kaolin, starch, smectite clays, chemically modified magnesium aluminum silicate, organically modified montmorillonite clay, hydrated aluminum silicate, fumed silica, aluminum starch octenyl succinate and mixtures thereof.

An alternative formulation for topical delivery is an array of microneedles. Microneedles (MN), as used herein, refers to an array comprising a plurality of micro-projections, generally ranging from about 25 to about 2000 μm in length, which are attached to a base support. An array may comprise 10², 10³, 10⁴, 10⁵ or more microneedles, and may range in area from about 0.1 cm² to about 100 cm². Application of MN arrays to biological membranes creates transport pathways of micron dimensions, which readily permit transport of macromolecules such as large polypeptides. In some embodiments of the invention, the microneedle array is formulated as a transdermal drug delivery patch. MN arrays can alternatively be integrated within an applicator device which, upon activation, can deliver the MN array into the skin surface, or the MN arrays can be applied to the skin and the device then activated to push the MN through the SC.

Various materials have been used for microneedles. For example, biodegradable materials into which the therapeutic agent, e.g. Benzamil, can be incorporated are of interest. Such materials include various biodegradable or biocompatible polymers or cross-linked monomers, as known in the art. The dose of agent to be delivered will vary, and may range from at least about 1 ng/microneedle array, at least about 10 ng, at least about 0.1 μg, at least about 1 μg, at least about 10 μg, at least 0.1 mg, at least 1 mg, or more in a single array. MNs may be fabricated with a wide range of designs (different sizes and shapes) and different types (solid, hollow, sharp, or flat), and may be in-plane and/or out-of-plane.

Polymeric MNs can provide biocompatibility, biodegradability, strength, toughness, and optical clarity. To accurately produce the micro-scale dimensions of polymer MNs, a variety of mould-based techniques, such as casting, hot embossing, injection molding, and investment molding may be used, e.g. beveled-tip, chisel-tip, and tapered-cone polydimethylsiloxane (PDMS) molds. Polymeric materials of interest for fabrication include without limitation; poly (methylmetha-acrylate) (PMMA), poly-L-lactic acid (PLA), poly-glycolic acid (PGA), and poly-lactic-co-glycolic acid (PLGA), cyclic-olefin copolymer, poly (vinyl pyrrolidone), and sodium carboxymethyl cellulose. Sugars have also been used to fabricate the MNs, such as galactose, maltose, aliginate, chitosan, and dextrin. Materials may be cross-linked through ion exchange, photo-polymerization, and the like.

In other embodiments, a topical formulation is provided as a transdermal patch. Medical dressings suitable for formulation in a transdermal patch can be any material that is biologically acceptable and suitable for placing over the skin. In exemplary embodiments, the support may be a woven or non-woven fabric of synthetic or non-synthetic fibers, or any combination thereof. The dressing may also comprise a support, such as a polymer foam, a natural or man-made sponge, a gel or a membrane that may absorb or have disposed thereon, a therapeutic composition. A gel suitable for use as a support is sodium carboxymethylcellulose 7H 4F, i.e. ethylcellulose.

For example, hydrocolloids (eg, RepliCare, DuoDERM, Restore, Tegasorb), which are combinations of gelatin, pectin, and carboxymethylcellulose in the form of wafers, powders, and pastes; some have adhesive backings and others are typically covered with transparent films to ensure adherence. Alginates (polysaccharide seaweed derivatives containing alginic acid), which come as pads, ropes, and ribbons (AlgiSite, Sorbsan, Curasorb), are indicated for extensive exudate and for control of bleeding after surgical debridement. Foam dressings (Allevyn, LYOfoam, Hydrasorb, Mepilex, Curafoam, Contreet) are useful as they can handle a variety of levels of exudate and provide a moist environment for healing. Those with adhesive backings stay in place longer and need less frequent changing.

In some embodiments, a transdermal patch comprises permeation enhancer, e.g. transcutol, (diethylene glycol monoethyl ether), propylene glycol, dimethylsulfoxide (DMSO), menthol, 1-dodecylazepan-2-one (Azone), 2-nonyl-1,3-dioxolane (SEPA 009), sorbitan monolaurate (Span20), and dodecyl-2-dimethylaminopropanoate (DDAIP), which may be provided at a weight/weight concentration of from about 0.1% to about 10%, usually from about 2.5% to about 7.5%, more usually about 5%.

Transdermal patches may further comprise additives to prevent crystallization. Such additives include, without limitation, one or more additives selected from octyldodecanol at a concentration of from about 1.5 to about 4% w/w of polymer; dextrin derivatives at a concentration of from about 2 to about 5% w/w of polymer; polyethylene glycol (PEG) at a concentration of from about 2 to about 5% w/w of polymer; polypropylene glycol (PPG) at a concentration of from about 2 to about 5% w/w of polymer; mannitol at a concentration of from about 2 to about 4% w/w of polymer; Poloxamer 407, 188, 401 and 402 at a concentration of from about 5 to about 10% w/w of polymer; and Poloxamines 904 and 908 at a concentration of from about 2 to about 6% w/w of polymer.

Polyvinylpyrrolidine (PVP) may also be included in a transdermal patch formulation, for example at a concentration of from about 5 wt % to about 25 weight %, about 7 wt % to about 20 wt %, about 8 wt % to about 18 wt %, about 10 wt % to about 16 wt %, about 10 wt %, about 12 wt %, about 14 wt %, about 16 wt %.

Emulsifiers which may be used include glyceryl stearate, polysorbate 60, PEG-6/PEG-32/glycol stearate mixture, etc. Solvents which may be used include the lower alcohols, in particular ethanol and isopropanol, and propylene glycol.

Hydrophilic gelling agents include carboxyvinyl polymers (carbomer), acrylic copolymers such as acrylate/alkylacrylate copolymers, polyacrylamides, polysaccharides, such as hydroxypropylcellulose, natural gums and clays, and, as lipophilic gelling agents, representative are the modified clays such as bentones, fatty acid metal salts such as aluminum stearates and hydrophobic silica, or ethylcellulose and polyethylene.

Therapeutic formulations for treatment of psoriasis with an ENAC blocker, e.g. Benzamil, can be used alone or in combination with an additional agent, e.g., a therapeutic agent, said additional agent being selected by the skilled artisan for its intended purpose. For example, the additional agent can be a therapeutic agent art-recognized as being useful to treat psoriasis. The agents set forth below are illustrative for purposes and not intended to be limited. The combinations which are part of this invention can be an ENAC blocker and at least one additional agent selected from the lists below. The combination can also include more than one additional agent, e.g., two or three additional agents if the combination is such that the formed composition can perform its intended function.

Additional therapeutic agents include, without limitation, methotrexate, 6-MP, azathioprine sulphasalazine, mesalazine, olsalazine chloroquinine/hydroxychloroquine, pencillamine, aurothiomalate (intramuscular and oral), azathioprine, colchicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, etc., phosphodiesterase inhibitors, adensosine agonists, antithrombotic agents, complement inhibitors, adrenergic agents, agents which interfere with signaling by proinflammatory cytokines such as TNF.alpha. or IL-1 (e.g. IRAK, NIK, IKK, p38 or MAP kinase inhibitors), IL-1.beta. converting enzyme inhibitors (e.g., Vx740), anti-P7s, p-selectin glycoprotein ligand (PSGL), TNF.alpha. converting enzyme (TACE) inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors and the derivatives p75TNFRIgG and p55TNFRIgG, sIL-1RI, sIL-1RII, sIL-6R, soluble IL-13 receptor (sIL-13)) and anti-inflammatory cytokines (e.g. IL-4, IL-10, IL-11, IL-13 and TGFβ). In some embodiments the dose of the additional therapeutic agent when co-formulated with an ENAC blocker is lower than the conventional dose. In some embodiments, Benzamil is co-formulated with a glucocorticoid.

Treatment with an ENAC blocker can also be combined with PUVA therapy. PUVA is a combination of psoralen (P) and long-wave ultraviolet radiation (UVA) that is used to treat many different skin conditions. In still another embodiment, the compositions of the invention are administered with excimer laser treatment for treating psoriasis.

Treatment for psoriasis often includes a topical corticosteroids, vitamin D analogs, and topical or oral retinoids, or combinations thereof. In one embodiment, an ENAC blocker is administered in combination with or the presence of one of these common treatments.

The composition can be packaged in any suitable container to suit its viscosity and intended use. The invention accordingly also provides a closed container containing a therapeutically acceptable composition as herein defined.

The pharmaceutical compositions of the invention may include a “therapeutically effective amount” or a “prophylactically effective amount”. A “therapeutically effective amount” refers to an amount effective, at dosages and for periods of time necessary, to achieve the desired therapeutic result. A therapeutically effective amount may vary according to factors such as the disease state, age, sex, and weight of the individual, and the ability to elicit a desired response in the individual. A therapeutically effective amount is also one in which any toxic or detrimental effects are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation.

In one embodiment, the dose is administered to the subject upon a flare of psoriasis. In another embodiment, the dose is administered to the subject prior to a flare of psoriasis.

The flare of psoriasis may be monitored by determining a subject's Psoriasis Area and Severity Index (PAST), e.g., PASI 100 response, PASI 90 response, PASI 75 response, PASI 50 response, the PASI response of a single body region, two body regions, three body regions, or four body regions, e.g., trunk, lower extremities, upper extremities, or head and neck. Alternatively, the flare of psoriasis may be monitored by determining a subject's Physician's Global Assessment (PGA) rating.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

### Methods of Use

The diagnosis of psoriasis is usually based on the appearance of the skin. Additionally a skin biopsy, or scraping and culture of skin patches may be needed to rule out other skin disorders. An x-ray may be used to check for psoriatic arthritis if joint pain is present and persistent.

A composition comprising an effective dose of an Rac1 inhibitor, optionally combined with additional therapeutic agents, is provided to an individual with psoriasis. The administration can be oral, parenteral, topical, etc. In some embodiments topical is preferred. The dosing and periodicity of administration is selected to provide for therapeutic efficacy.

In one embodiment, the subject achieves at least a PGA score of 0 or 1. In one embodiment, the subject achieves at least a PASI 75 response. In one embodiment, the subject achieves at least a PASI 90 response. In one embodiment, the subject achieves at least a PASI 100 response. In one embodiment, the subject maintains the PGA score of 0 or 1 during treatment. In one embodiment, the subject maintains the PASI 75 response during treatment. In one embodiment, the subject maintains the PASI 90 response during treatment.

In one embodiment, the subject achieves a PGA score of 0 or 1, e.g., by about week 12. In one embodiment, the subject achieves at least a PASI 75 response, e.g., by about week 12. In one embodiment, the subject achieves at least a PASI 90 response, e.g., by about week 12. In one embodiment, the subject achieves at least a PASI 100 response, e.g., by about week 12.

In one embodiment, the subject maintains the PGA score of 0 or 1 through the duration of treatment. In one embodiment, the subject maintains the PASI 75 response through the duration of treatment. In one embodiment, the subject maintains the PASI 90 response through the duration of treatment.

In certain embodiments of the foregoing aspects, the subject or population of subjects achieves (i) an improvement in a Dermatology Life Quality Index (DLQI) score or mean Dermatology Life Quality Index (DLQI) score of at least about −9; (ii) an improvement in a Short Form 36 Health Survey Physical Component Summary (PCS) score or mean Physical Component Summary (PCS) score of at least about 2; (iii) an improvement in a Short Form 36 Health Survey Mental Component Summary (MCS) score or mean Short Form 36 Health Survey Mental Component Summary (MCS) score of at least about 4; (iv) an improvement in a visual analog scale score or mean visual analog scale score for psoriasis-related pain (VAS-Ps) of at least about −25; (v) an improvement in a visual analog scale score for psoriatic arthritis-related pain (VAS-PsA) or mean visual analog scale score for psoriatic arthritis-related pain (VAS-PsA) of at least about −32; and/or (vi) a minimum clinically important difference (MCID) response rate for psoriasis-related pain (VAS-Ps) of at least about 60%.

In various aspects, the invention is directed to a method of treating psoriasis in a population of subjects, wherein the population of subjects achieves (i) a minimum clinically important difference (MCID) response rate for Dermatology Life Quality Index (DLQI) of at least about 70% by about week 12; (ii) a minimum clinically important difference (MCID) response rate for Dermatology Life Quality Index (DLQI) of at least about 81% by about week 52; (iii) a minimum clinically important difference (MCID) response rate for Total Activity Impairment (TAI) of at least about 45% by about week 12; and/or (iv) a minimum clinically important difference (MCID) response rate for Total Activity Impairment (TAI) of at least about 57% by about week 52. In one embodiment, the antibody, or antigen-binding portion thereof, is administered once every four weeks. In another embodiment, the antibody, or antigen-binding portion thereof, is administered once every 12 weeks.

In certain embodiments of the various aspects of the invention, the subject achieves a Nail Psoriasis Severity Index (NAPSI) score of about 2.1 or less. In certain embodiments, the subject achieves a Nail Psoriasis Severity Index (NAPSI) score of about 2.1 or less by about week 24. In related embodiments of the various aspects of the invention, the subject achieves a Nail Psoriasis Severity Index (NAPSI) score of about 1.2 or less. In certain embodiments, the subject achieves a Nail Psoriasis Severity Index (NAPSI) score of about 1.2 or less by about week 52.

### EXAMPLES

The following examples are offered by way of illustration and not by way of limitation.

### Example 1

### Rac1 Drives Pathologic Epidermal-Immune Interactions

Epidermal-immune interactions governing epidermal tissue homeostasis are altered in psoriasis, an inflammatory disease affecting one in thirty adults. Here, we characterize Rac1 as a key mediator of this process. Rac1 activation was consistently elevated in psoriatic epidermis and primary psoriatic human keratinocytes (PHKC). Mice expressing K14 driven V12Rac1 activated mutant closely mimicked human psoriasis, requiring an intact immune system for disease progression. Mouse and human psoriatic skin showed similar Rac1 dependent signaling and transcriptional overlap of epidermal and immune pathways. PHKC displayed Rac1-dependent upregulation of pro-inflammatory cytokines following immunocyte coculture, mimicked by overexpressing V12Rac1 in normal human keratinocytes. Modulating Rac1 activity perturbed differentiation, proliferation and inflammatory pathways including STAT3, NFκB and ZNF750. Reconstructed patient PHKC/immunocyte xenografts showed psoriasiform hyperplasia and inflammation in vivo, which was abolished by inhibiting Rac1 activity in PHKC. These studies implicate Rac1 as a novel therapeutic psoriasis target and a key orchestrator of pathologic epidermal-immune interactions.

Interactions between cutaneous epithelia and the immune system are vital for maintaining epidermal tissue homeostasis, skin barrier integrity and immunocyte responses. Disruption of this interplay can lead to psoriasis, a chronic inflammatory disorder affecting 3% of all individuals. Psoriasis is characterized by pruritic, disfiguring skin lesions, arthritis in up to 30% of cases, and increased risk of myocardial infarction and stroke.

Immune target identification has led to new immune based biologic therapies. However potential risks of systemic immunosuppressive therapy such as serious infections and tuberculosis activation along with a lack of full understanding of long term biologic immunosuppressive risks, make the search for alternatives to long term systemic immunosuppression highly relevant, especially in the treatment of a lifelong disorder such as psoriasis.

Despite the longstanding recognition of epidermal dysfunction as an intrinsic feature of psoriasis, and the implication of epidermal pathways in analysis of psoriasis genome wide association studies, significant gaps still exist in our understanding of the role of the epidermis in psoriasis pathogenesis, which has limited development of epidermal targeted therapies.

One aspect of the disease process which has confounded mechanistic studies to date is the observation that psoriasis is not only genetic in origin, but also is highly responsive to various environmental stimuli, with cutaneous wounding, known as the Koebner phenomenon and Group A streptococcal (GAS) infection, being the two most well-known psoriasis triggers. How seemingly distinct environmental triggers can interact with predisposing genetic factors to activate psoriasis has not been answered and a unifying model incorporating both genetic and environmental influences in psoriasis pathogenesis has not yet been established. However, one potential clue lies in the epidermal activation of the small GTPase Rac1. Rac1 activation is known to be triggered in response to extracellular environmental signals, a process believed to promote epidermal proliferation required for wound repair. Furthermore, the binding of streptococcal capsular polysaccharide with the keratinocyte CD44 cell surface receptor strongly induces epidermal Rac1 activation. These findings, appearing to link together diverse external psoriasis triggers led us to examine the role of epidermal Rac1 activation as an etiologic factor in psoriasis pathogenesis.

### Results

Epidermal Rac1 Hyperactivation in Human Psoriasis.

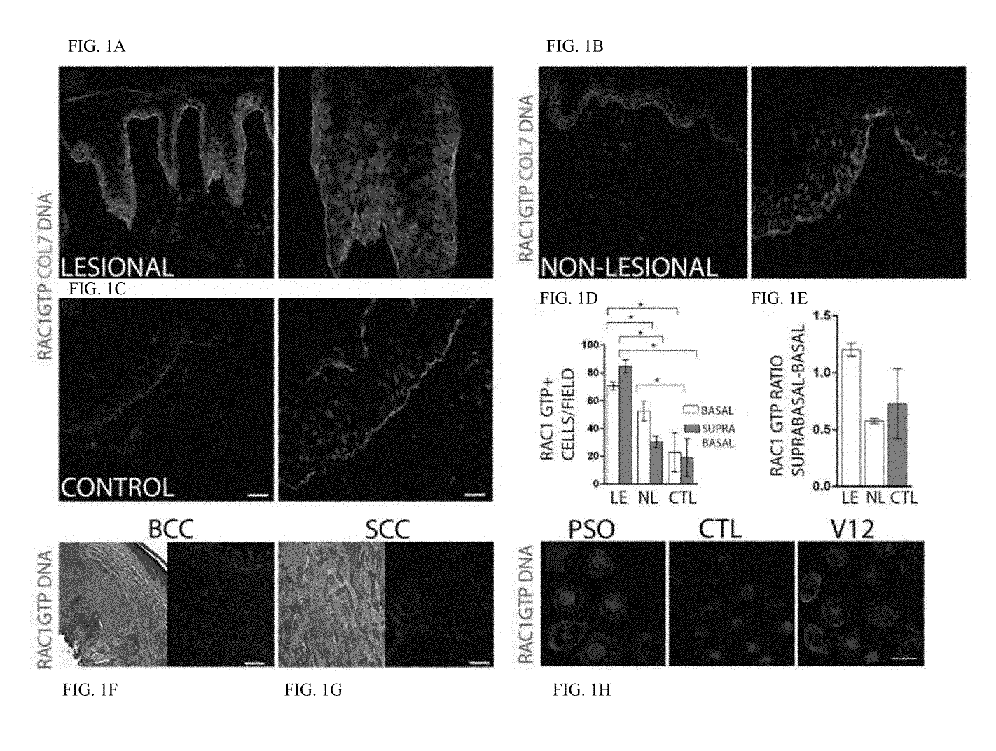

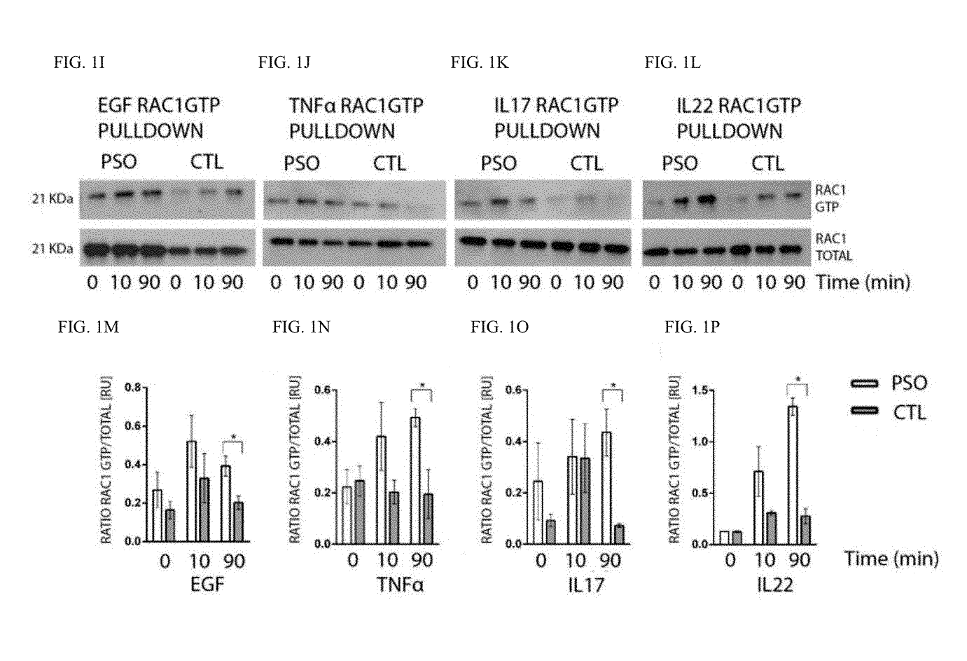

To examine the activation state of Rac1, psoriatic skin was tested by indirect immunofluorescence microscopy (IDIF) using a Rac1GTP active specific mAb. Abnormally high Rac1 activation was seen (FIG. 1A-C, D-E) in suprabasal and basal lesional epidermis (n=19) and in basal nonlesional epidermis (n=4). However, Rac1 activation was not elevated in basal- (n=3) or squamous cell carcinoma (n=3), or in a mouse contact dermatitis model (FIG. 1F, G). In contrast, another Rho GTPase, RhoA, showed no increased activation in psoriatic skin. Specificity of these antibodies was confirmed using organotypic 3D skin equivalents with keratinocytes overexpressing activated mutants of V12Rac1, V14RhoA or LacZ control.

To further investigate the significance of epidermal Rac1 activation in psoriasis, primary psoriatic human keratinocytes (PHKC) from non-lesional skin, cultured in serum free medium (n=7), showed marked, cytoplasmic Rac1GTP distribution, compared to a reduced and peripheral distribution in primary normal human (n=4) keratinocytes (NHKC) (FIG. 1H). NHKC overexpressing activated (V12) Rac1 mutant (12) (Rac1NHKC) showed a similar intracellular distribution to PHKC. Following growth factor starvation, additions of psoriasis related stimuli EGF, TNFα, IL17A/F, IL22 or GAS capsular extract (but not IL6, data not shown) triggered Rac1 hyperactivation in PHKC compared to NHKC (FIG. 1I-P). In total, all PHKC tested consistently showed marked Rac1 hyperactivation in response to exogenous stimuli.

Epidermal Rac1 Hyperactivation in Mice Closely Mimics Human Psoriasis.

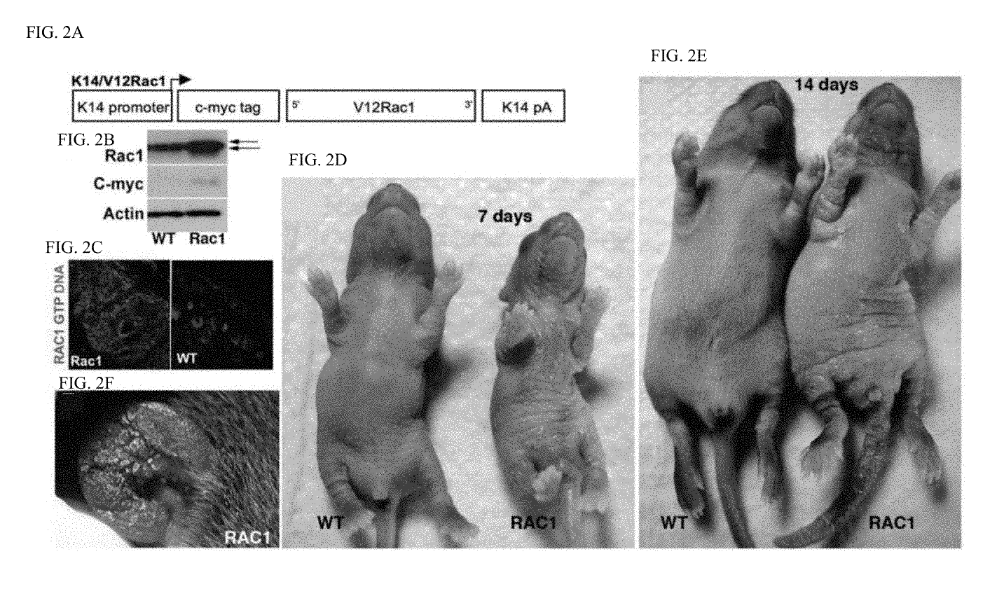

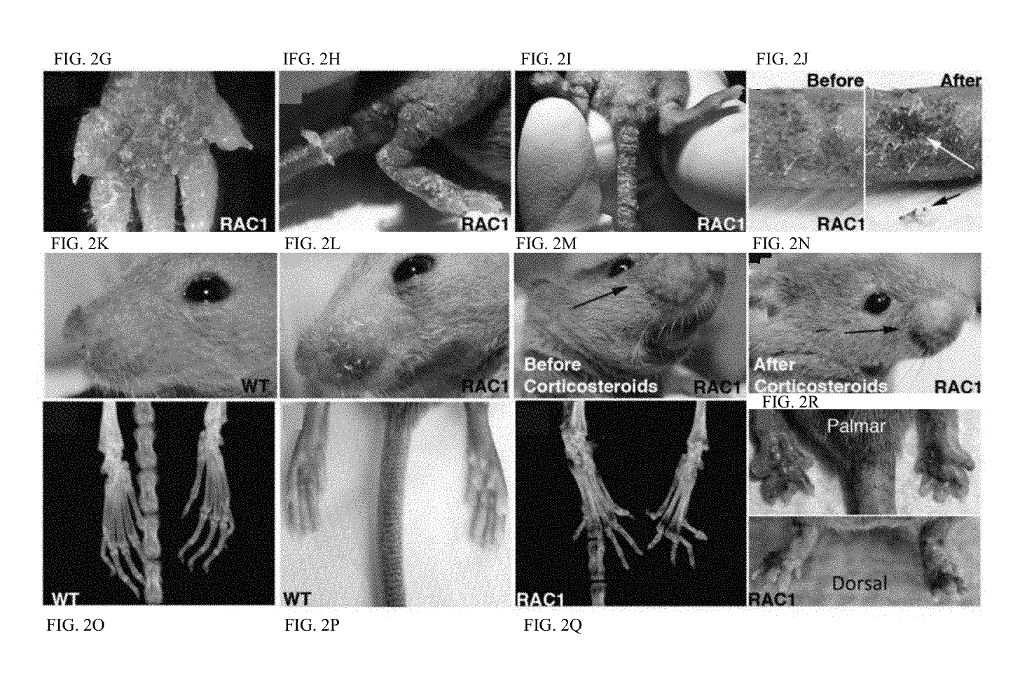

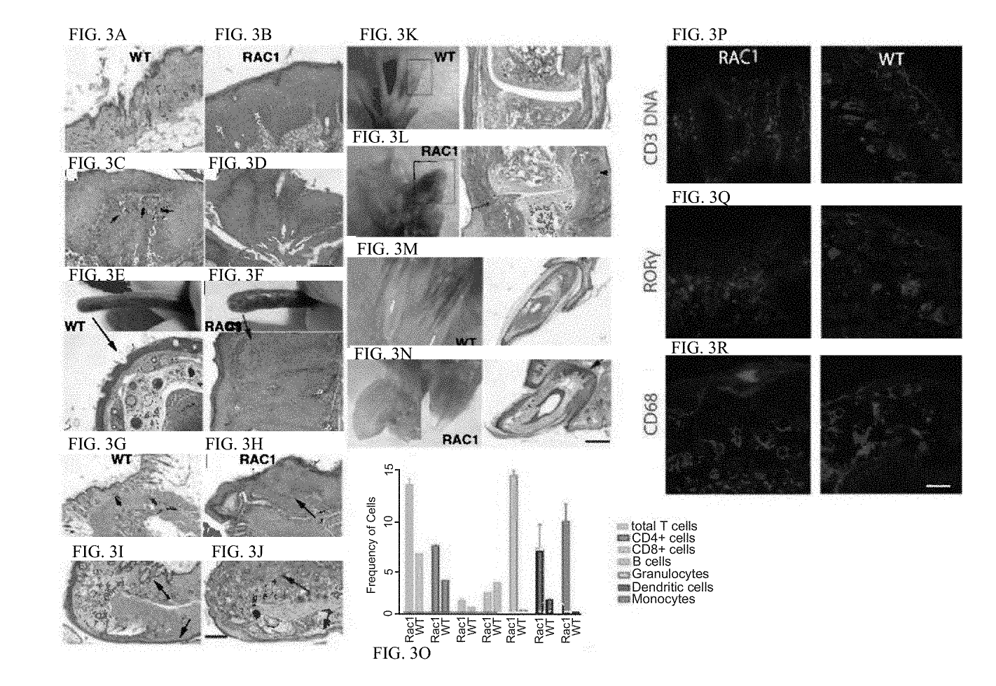

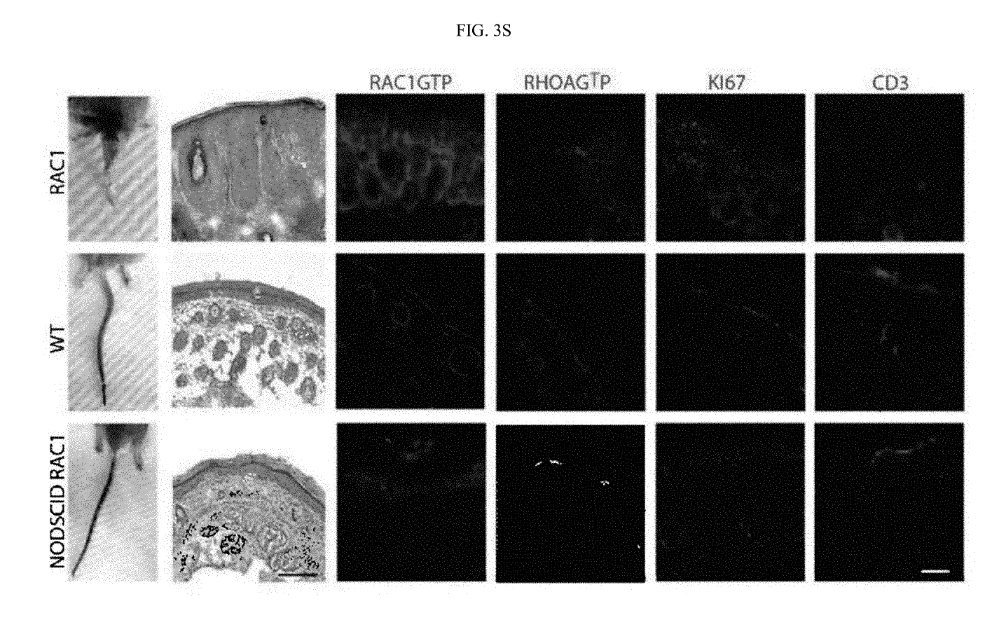

To determine the role of epidermal Rac1 activation in psoriasis, a keratin 14 (K14) driven V12 activated Rac1 mutant (FIG. 2A-C) was expressed in transgenic mice, and validated by immunoblot (V12Rac1 band, upper arrow) and IDIF. Wildtype (WT) control skin showed minimal Rac1 activation, except focally at wound edges. Erythematous scaling skin lesions developed by 7 days (FIG. 2D), thickening by 14 days, commonly localizing to ears, paws, tail and snout (FIG. 2F-M). Rac1 mice were smaller than wild type siblings. Lesions showed hemorrhage (Auspitz sign) following scale removal (FIG. 2J). Rac1 mothers often bit their pups' whiskers and snout hair (possibly to decrease nursing related itching). Mutant (but not normal) pups developed psoriasis like lesions in response to this trauma, similar to the Koebner phenomenon of human psoriasis (FIG. 2K, L). Skin lesions improved following topical corticosteroid treatment (FIG. 2M, N). By one month, most mice developed erythema and edema of the tail and paws, however ˜50% showed a pronounced mutilating arthropathy, with bony paw deformations (FIG. 20-R) and/or partial tail auto-amputation (FIG. 3S, top left).

Lesional Rac1 skin showed pronounced psoriasiform hyperplasia, hypogranulosis, mixed inflammatory infiltrates (FIG. 3A, B), dilated vessels in dermal papillae (FIG. 3C, arrows) and marked parakeratosis (FIG. 3D). Wound-induced psoriasiform hyperplasia typically followed tail snip for genotyping (FIG. 3E, F). Mucosa (but not perimucosal skin) was spared from proliferative/inflammatory changes, as demonstrated in anus (FIG. 3G, H), and eyelids (FIG. 31, J, lower arrows). Rac1 mice with joint involvement showed neutrophillic infiltrates near joint spaces (FIG. 3L, arrow). Nail changes ranged from mild to severe with nail matrix showing psoriasiform hyperplasia (FIG. 3M-N, arrow).

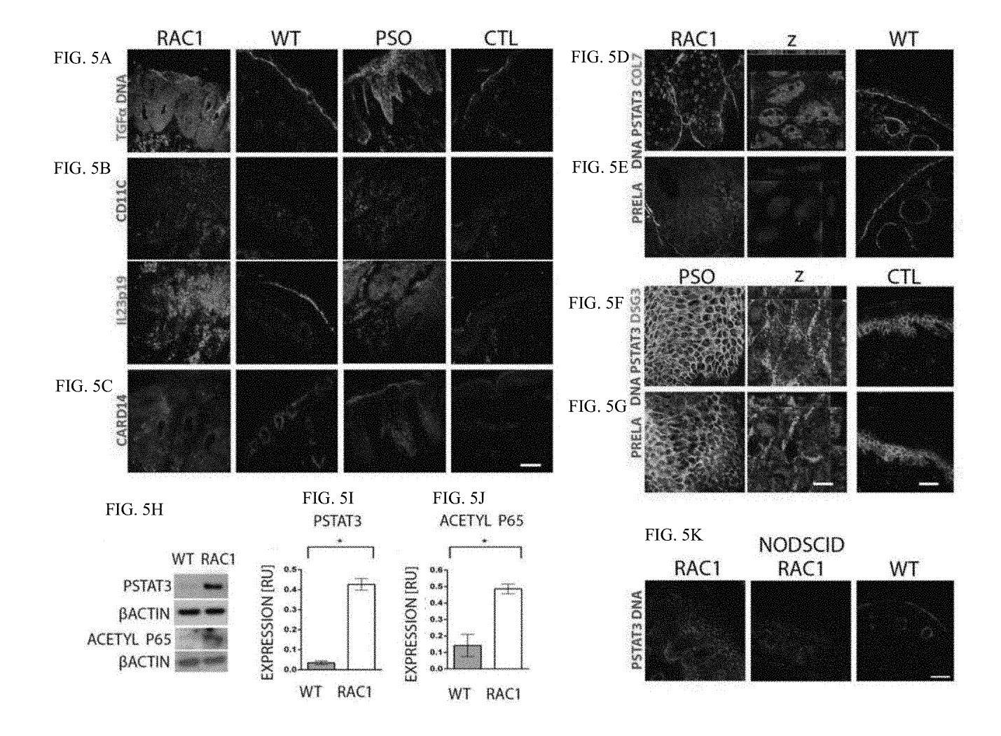

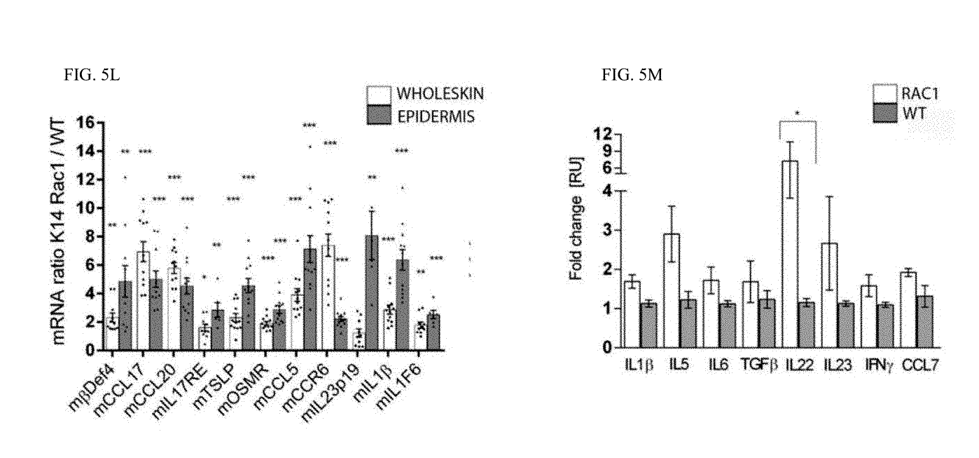

Lesional Rac1 skin showed increased CD4+ and CD8+ lymphocytes, neutrophils and dendritic cells (DC) by FACS analysis (FIG. 30). Increased CD3+ lymphocytes expressing IL17 and RORγ, CD11c+ DCs, and CD68+ cells (Munro's micro abscesses) in the stratum corneum were noted by IDIF (FIG. 3P-R, FIG. 5B). Suprabasilar proliferation (by Ki67) was prominent (FIG. 3S). Both dermal CD11c+ and epidermal cells demonstrated IL23 expression in lesional skin (FIG. 5B). V12Rac1 transgene produced a similar psoriatic phenotype across CBA (FIG. 2, 3) BALB/c and C57BL/6 backgrounds, however backcrossing to a NOD/SCID strain lacking functional lymphocytes, strikingly reduced epidermal thickness and proliferation (FIG. 3S) as well as tail or joint abnormalities, despite persistent Rac1 activity in NOD-SCID V12 skin. Similarly, cyclosporin A treatment significantly reduced epidermal thickness, proliferation and T-cell infiltration. In total, these results show that epidermal Rac1 activation closely mimicked the cutaneous and rheumatologic phenotype of human psoriasis, but only in the presence of an intact immune system.

Transcriptional Profile of Rac1 Murine Skin and Overlapping Signatures in Human Psoriatic Lesional Skin.

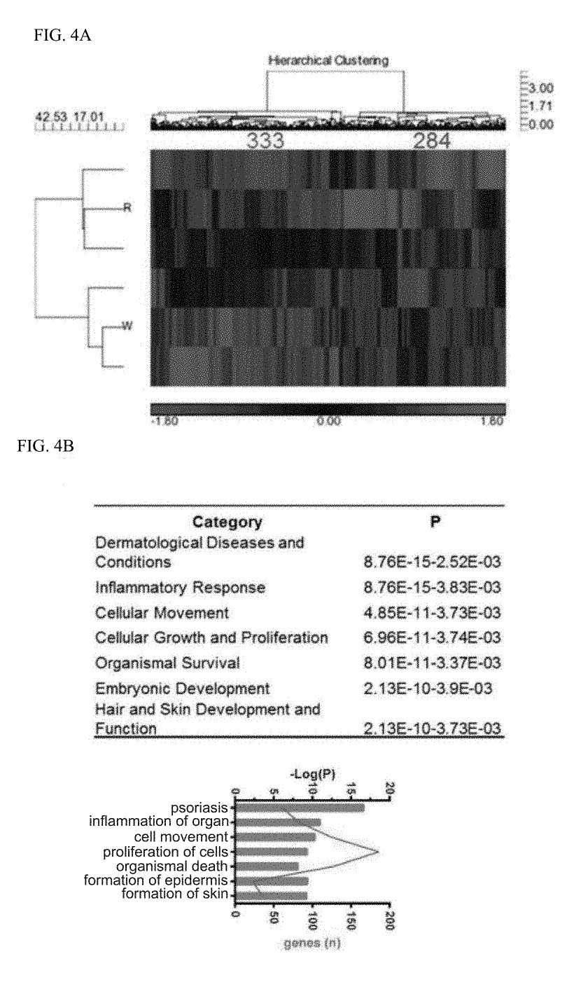

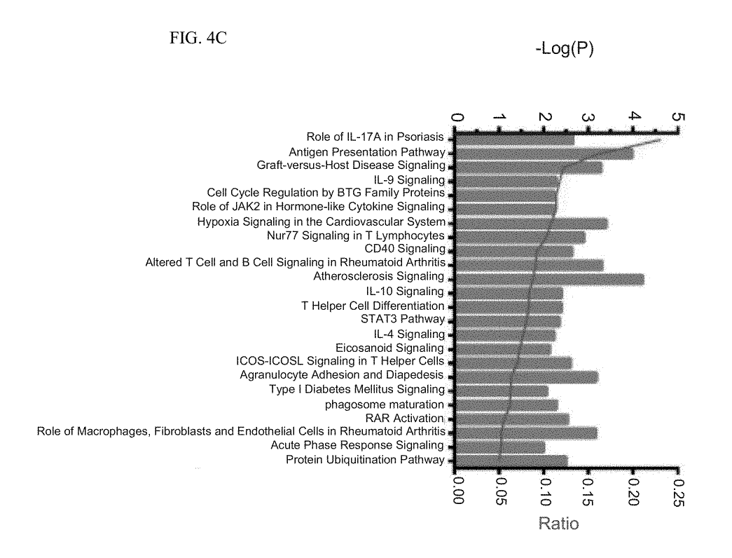

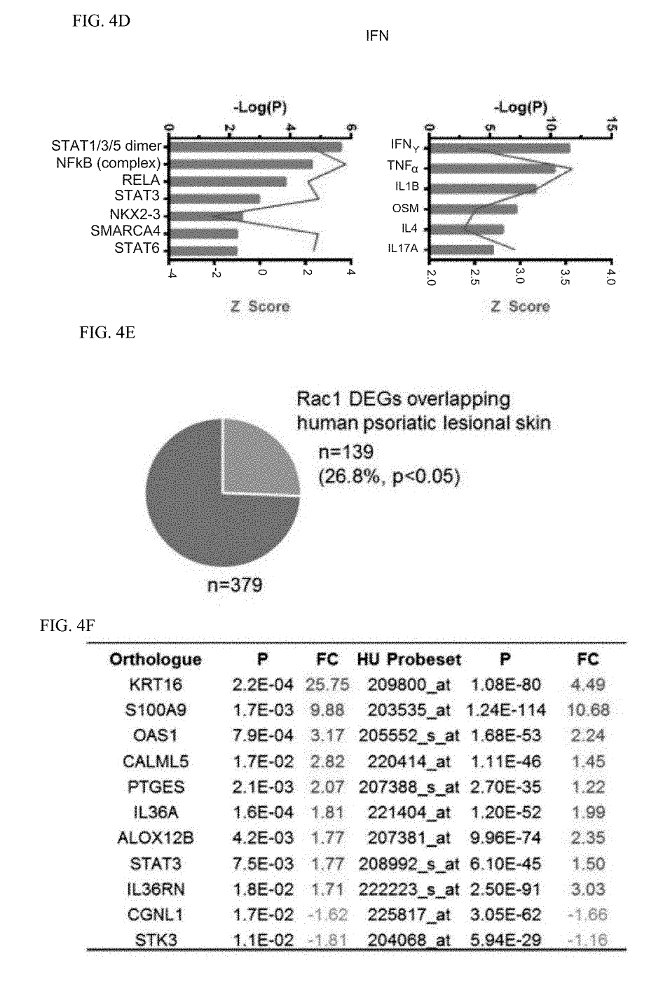

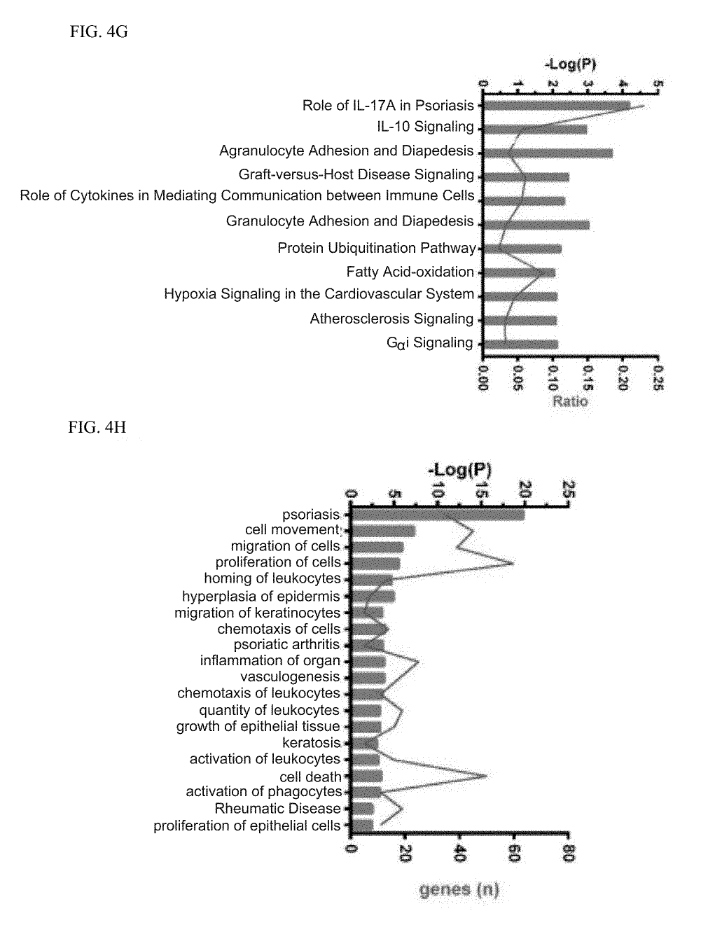

Transcriptional analysis of day 7 Rac1 mouse skin compared to WT littermates identified 284 (46%) induced and 333 (54%) repressed differentially expressed genes (DEGs) (FIG. 4A), annotated to dermatological diseases and conditions (psoriasis); inflammatory response (inflammation of organ); cellular movement; cellular growth and proliferation (proliferation of cells); organismal survival (organismal death) and embryonic development (formation of epidermis) (FIG. 4B). Enriched canonical pathways involved role of IL17A in psoriasis, antigen presentation pathway, CD40 signaling and altered T cell and B cell signaling in rheumatoid arthritis (FIG. 4C). Activation z scores of transcription factors (TFs) and cytokines included STAT3, NFκB, IFNγ, TNFα, IL1β and IL17A signaling (FIG. 4D). A human psoriasis dataset yielded significant enrichment for interferon, STAT3, NFκB and Rho family GTPase signaling (Rac1 but not RhoA). Merging Rac1 mouse skin orthologous DEGs (n=518) with this ranked human dataset demonstrated a significantly overlapping signature (n=139, p<0.05) (FIG. 4E), including KRT16, S100A9, IL36A/RN, STAT3 and CGLN1 (FIG. 4F). Enriched pathways encompassed role of IL17A in psoriasis (IL117RC, S100A8, S100A9), agranulocyte adhesion and diapedesis, role of cytokines in mediating communication between immunocytes, protein ubiquitination pathway and atherosclerosis signaling; and biological functions included psoriasis, migration, proliferation, leukocyte homing and psoriatic arthritis. IPA TF motif enrichment included associations to JUN/FOS, STAT3, IRF1/3/7 and NFκB complex (REL/RELA/RELB/NFκB1/NFκB2).

Most significant non-overlapping signatures in Rac1 mouse skin included antigen presentation Pathway, RAR activation, CD40 signaling and atherosclerosis signaling, whereas most significantly induced non-overlapping signatures in human psoriatic skin contained protein ubiquitination pathway, cell cycle regulation, molecular mechanisms of cancer and interferon signaling. In silico mapping of psoriasis susceptibility genes to interactions with each other and Rac1, implicated a network including JAK/STAT, NFκB, Rac1 and IRF signaling. Transcriptional overlap between Rac1 and other mouse models of psoriasis showed the most significant overlap with the K14AREG and K5STAT3C models. Enriched pathways included STAT3-, immune cell-IL22- and JAK signaling, whereas distinct signatures enriched in Rac1 skin included antigen presentation pathway, role of IL17 in psoriasis, atherosclerosis- and arthritis-associated pathways.

Rac1 murine skin mimics proliferative, proinflammatory and differentiation signaling seen in human psoriasis. Rac1 mouse and human psoriatic epidermis showed similar proliferative and inflammatory protein expression. Mouse and human skin showed increased TGFα, CARD14, and IL23p19 (FIG. 5A-C) as well as activation of STAT3 and NFκB (FIG. 5D-G), verified by nuclear localization and immunoblot (FIG. 5D-J), however epidermal pSTAT3 was strongly reduced in epidermis of immunodeficient V12 Rac1 mice (FIG. 5K). Rac1 skin showed increased expression of psoriasis associated proteins β defensins, CCL17, CCL20, CCL5, CCR6, TSLP, oncostatin M receptor, IL23p19 and IL1F6 (IL36a) (FIG. 5L).

To assess spatiotemporal relationships of psoriasis associated chemokines in inflamed Rac1 skin, we compared epidermis to whole skin between Rac1 and WT at 1 day of age, preceding immune cell infiltration and psoriasiform hyperplasia, (pre-lesional skin) compared to 7 day old pup skin, when marked psoriasform hyperplasia and immune cell infiltration was evident. We found a number of cytokines significantly increased in pre-lesional epidermis, including CCL2, CCL5, CCL20, CXCL1, β4-Defensin, CXCL11, OSMR and TSLP. By one week of age, there was an additional increase in CCL20 and IL1-6. Pre-lesional whole skin had a marked increase of cytokines such as CCL2, CCL5, CXCL10 and CXCL11, whereas lesional skin also included significantly upregulated mRNA levels of CXCL2 and IL1-6. Due to the marked joint inflammation in Rac1 mice, we tested whether Rac1 activation in the skin could be associated with induction of a systemic inflammatory response. Luminex of mouse sera revealed that 3 week old Rac1 mouse sera contained significant increases in psoriasis associated cytokines IL-22 (˜7 fold) and increased IL-23 (—2.5 fold) compared to WT (FIG. 5M).

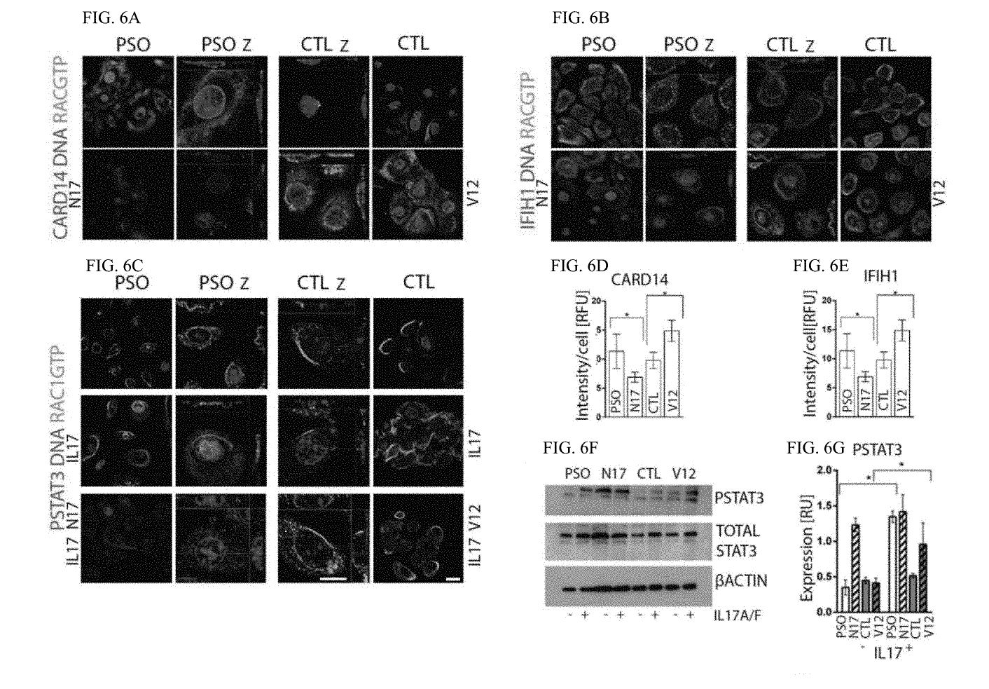

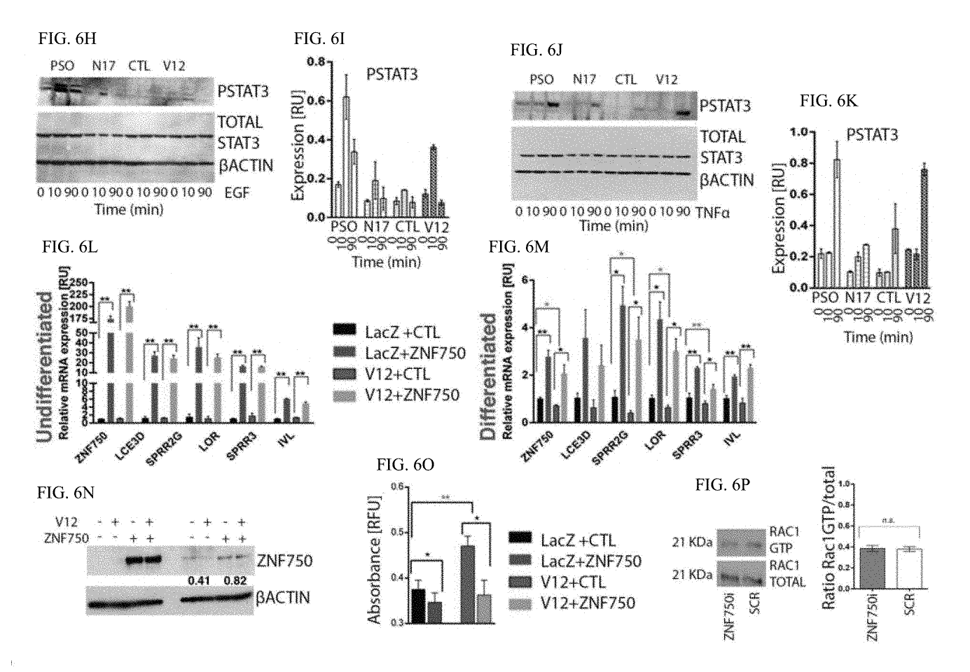

Rac1 activation promotes proliferation and inflammation related signaling in primary human psoriatic keratinocytes and xenografts. We next validated psoriasis related proliferation and inflammation regulators involving NFκB and STAT3 from our bioinformatics analysis for dependence on Rac1 activation. We found CARD14 nuclear rim and IFIH1 nuclear localization were increased in PHKC compared to NHKC, however, expression of dominant negative (N17) Rac1 mutant in PHKC reduced CARD14/IFIH1 localization. Conversely, expression of V12Rac1 in NHKC increased CARD14 and IFIH1 nuclear rim and nuclear localization (FIG. 6A, B, D, E). As our results indicated immune derived factors were required for activation of persistent phosphorylated STAT3 in V12 Rac1 mouse skin, we assayed how Rac1-activating cytokines (FIG. 1I-K) affect this process. IL17 A/F addition increased nuclear localization of STAT3 (in PHKC compared to NHKC (FIG. 6C). Notably, N17Rac1 overexpression led to a consistent cytosolic accumulation, and loss of nuclear translocation of PSTAT3 in PHKCs (FIG. 6C). Conversely V12Rac1 overexpression in NHKC increased IL17 associated STAT3 nuclear localization (FIG. 6C). We verified by immunoblot increased PSTAT3 in PSOKC compared to NHKCs (FIG. 6F, G), mimicked by V12 overexpression (FIG. 6F, G), and accumulation of PSTAT3 in N17PHKC, irrespective of IL17 stimulation. As the overlapping signature between V12Rac1 mouse and human psoriatic skin implicate IL17RC, we verified the presence of this signaling axis in keratinocytes. We detected IL17RC expression by psoriasis and control keratinocytes, and the IL17R adaptor TRAF3IP2 in lesional mouse and human psoriatic epidermis. EGF and TNFα also promoted STAT3 activation (FIG. 6H-K) and nuclear localization in PHKC, which was reduced following N17Rac1 overexpression. Conversely, V12Rac1 overexpression in NHKC increased STAT3 activation and nuclear localization following EGF or TNFα treatment. We also found a reduced accumulation of cytosolic PSTAT3 in N17 PHKC after 24 hours (FIG. 6H, J) compared to 48 hours (FIG. 6F), in agreement with sequestration of cytosolic PSTAT3 without active Rac1. Interestingly, while the effects of EGF on PHKC and V12Rac1NHKC STAT3 phosphorylation peaked early at 10 minutes, suggesting a direct Rac1 effect, effects of TNFα on STAT3 phosphorylation were noted only after 90 minutes (FIG. 6F), and after 24 hours for IL17, suggesting a delayed or indirect effect. Altogether, this suggests that active Rac1 may regulate these NFκB and STAT3-associated signaling events in psoriatic keratinocytes.

Given the perturbed differentiation and proliferation in Rac1 murine epidermis in vivo, we compared differentiation markers in V12Rac1NHKC to LacZNHKC, including the psoriasis susceptibility gene ZNF750. After confirming that ZNF750 upregulated LCE3D, SPRR2G, SPRR3 and IVL mRNA in both V12 and LacZ conditions (FIG. 6L) we induced differentiation in these cultures. We found modestly reduced ZNF750 mRNA (FIG. 6M) but markedly reduced protein (0.42/1) (FIG. 6N) in V12Rac1 compared to LacZ NHKC, accompanied by significant decreases in SPRR2G, SPRR3 and loricrin mRNA. Ectopic ZNF750 restored V12Rac1 induced repression to ˜80%, indicating additional post-translational downregulation of ZNF750 protein expression. V12Rac1NHKC showed increased proliferation compared to LacZNHKC, but ZNF750 induction abolished this difference (FIG. 6O). Rac1GTP pulldown of ZNF750-depleted NHKC did not activate Rac1 compared to scrambled control (FIG. 6P), excluding loss of ZNF750 as an activator of Rac1. These findings demonstrate one pathway whereby human keratinocyte-activation of V12Rac1 may inhibit differentiation pathways and promote proliferation through repressing ZNF750 transcription.

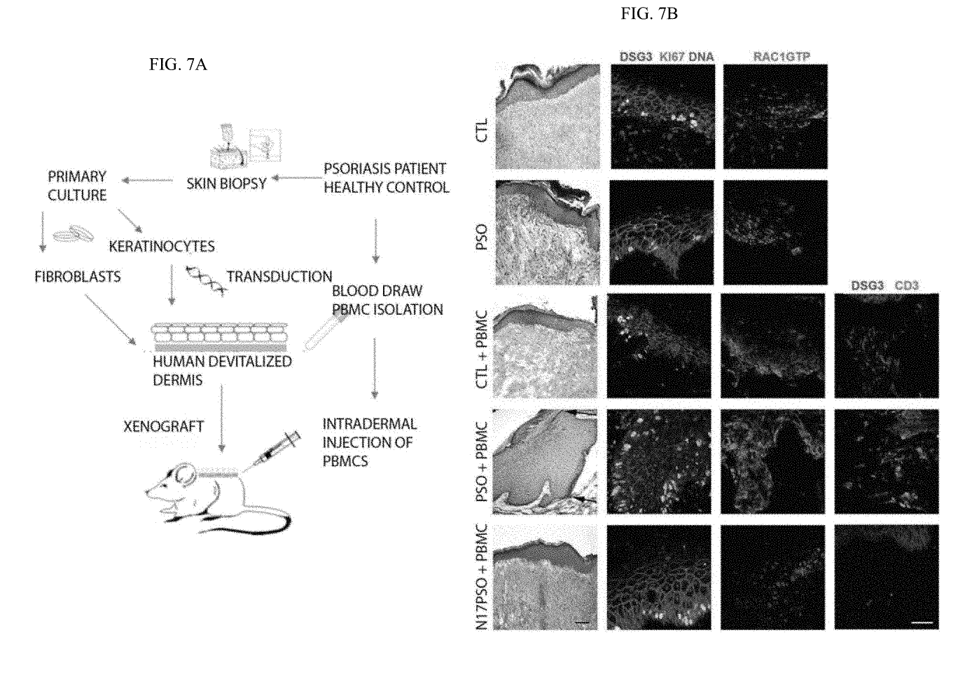

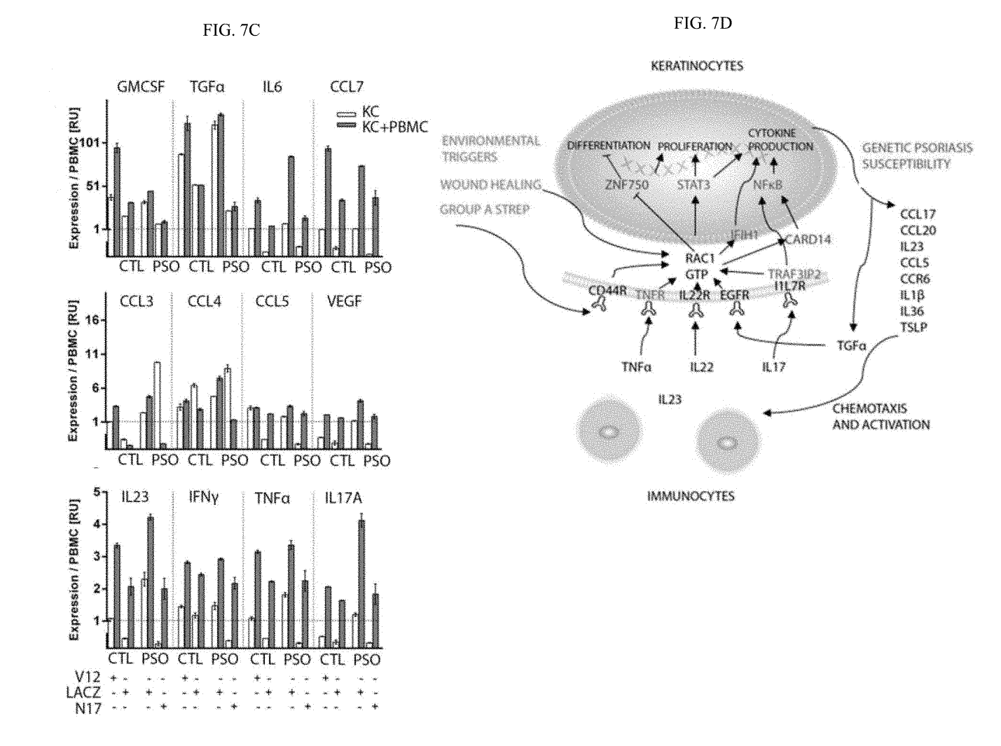

PHKC cultured atop devitalized dermis and xenografted to NOD/SCID mice showed epidermal thickness comparable to control NHKC xenografts, however striking psoriasisform hyperplasia and inflammatory infiltrates were noted in PHKC (but not in NHKC) xenografts following the injection of autologous PBMCs (FIG. 7A, B). Hyperplasia and infiltrates in PHKC xenografts were completely normalized following epidermal N17Rac1 overexpression. To evaluate the role of epidermal Rac1 in promoting inflammation, PHKC and NHKC were co-cultured in vitro with PBMCs, and cytokines in conditioned medium were assayed after 48 h. PHKC and V12Rac1NHKC co-cultures demonstrated elevated expression of an array of cytokines (including GMCSF, TGFα, IL6, CCL17, CCL3, CCL4, CCL5, VEGF, IL23, IFNγ, TNFα, and IL17) not seen in N17Rac1 PHKC and NHKC co-cultures (FIG. 7C). In total, these results demonstrate that the epidermal Rac1 signaling dictates both proliferative and immune related aspects of the psoriatic phenotype, but requires both intrinsic activation and immune-derived factors. A model explaining our findings suggest that Rac1 activation lies at the interface of a number of signaling pathways involving psoriasis genetic susceptibility loci (FIG. 7D).

Epidermal Rac1's central role in psoriatic epidermis, as summarized in FIG. 7D, suggests a close association with both environmental triggers as well as genetic psoriasis susceptibility factors. Through its effects on key transcription factors STAT3, ZNF750, IRFs and NFκB, Rac1 appears to inhibit epidermal differentiation, as well as promote epidermal proliferation and production of proliferative and proinflammatory molecules. Some of these secreted agents such as TGFα may act in an autocrine manner on keratinocyte receptors; however other proinflammatory agents induced by epidermal Rac1 activation likely promote both immune chemotaxis and differentiation, leading to increased local immune production of TNFα, IL23, IL22 and IL17. Through the ability of TNFα, IL17 and IL22 to promote further Rac1 activation, and the ability of Rac1 activation to induce proinflammatory cytokines, epidermal Rac1 activation appears to drive a positive feedback loop between the epidermis and immune system in promoting psoriasis pathogenesis.

Though epidermal Rac1 hyperactivation was common to human psoriasis, it was not seen in many other epidermal proliferative or inflammatory conditions studied. Moreover, since previous studies of Rac1 null mice showed normal epidermal proliferation and no inhibition of contact dermatitis associated inflammation, Rac1's role in epidermal proliferation/inflammation appeared distinct. However, we found reduced levels of the other Rho family GTPases RhoA and to a lesser extent CDC42 in Rac1 lesional and V12 Rac1PHKCs. This suggests an intimate relationship of RhoGTPase regulation in skin homeostasis. We also found an enrichment of RhoGDI signaling in human psoriatic skin. Controlled epidermal cytokine production and proliferation in acute wound healing contrasts with uncontrolled cytokine production and proliferation in psoriatic epidermis, in fact, some have described psoriasis as exaggerated wound healing. In a similar manner, controlled Rac1 activation in wound healing, contrasted with its wide distribution in lesional psoriatic epidermis.

Rac1 hyperactivation in human psoriasis appeared to occur in a cell autonomous fashion as third passage non-lesional PHKC, cultured for three passages in the absence of immunocytes, displayed marked Rac1 activation in response to diverse stimuli, including the known psoriasis therapeutic targets TNFα and IL17. Hyaluronate rich GAS capsule, known for its ability to evade immune detection, leukocyte phagocytosis and demonstrated in serum of patients with active infections, is a strong inducer of epidermal Rac1 activation with psoriatic keratinocytes showing especially high levels. GAS capsular antigen derived from the serum or local microbiota could play a role in triggering pathologic epidermal Rac1 activation during psoriasis flares. Although downstream effectors of Rac1 has been genetically associated with psoriasis (FIG. 7D) and could lead to feedback effects on Rac1 activity, our results show that genetic predisposition to upstream events causing Rac1 activation may be present in some of our psoriatic samples. Although we did not find a consistent deregulation in the Rac1 exchange factors ARHGEF6, TIAM1 or RacGAP1, others may be altered and provide insights into potential upstream signaling events.

Our transgenic Rac1 mouse studies demonstrate epidermal Rac1 hyperactivation is sufficient to promote disease activity in the skin, nails and joints closely mimicking human psoriasis clinically and histologically. Auspitz sign, Koebnerization, response to cyclosporine and topical corticosteroids as well as pattern of arthritis closely mimicked human psoriasis. Remission in Rac1 mutants following crossing with immunodeficient mice is consistent with the immune dependency of human psoriasis. Rac1 dependent expression of chemotactic cytokines CCL20, CCL17 and CCL5 and TH17 differentiation promoting cytokines IL23, IL1β, and IL6 in mouse epidermis (FIG. 5L) and/or human keratinocytes (FIG. 7C) suggests a role for epidermal Rac1 activation in immune recruitment and activation. Although the relative contributions of IL23 from keratinocytes and immune cell subsets were not compared, our results support additional contribution of IL23 by immune-stimulated psoriatic keratinocytes to the elevated levels of IL23 seen in psoriatic skin, in agreement with previous studies on human psoriatic keratinocytes and skin. We found a significant enrichment for Rac1-signaling in mRNA expression data from psoriatic skin, including KRT6A/16, S100A7/8/9, IL36A/IL36RN, STAT3 and CGLN1 (FIG. 4F); and overlapping pathway enrichment including local and systemic psoriasis associated pathways (FIG. 4G-H). Importantly, we demonstrate that sustained Rac1 activation in epithelia can drive systemic manifestations and activate normal immune cells, which may have implications for other disease states implicating autoimmunity. The role of epidermal Rac1 activation in the promotion of an epidermal-immune feedback loop provides an interesting contrast with activating mutations in other GTPases such as Ras which have been associated with epidermal neoplasms. One key difference between these two processes may be that Rac1 activation requires immune participation to promote proliferation, whereas Ras activation does not.

Rac1 dependent activation of the inflammatory regulator NFκB was seen in both Rac1 mouse and human psoriatic epidermis. The psoriasis-associated NFκB activator CARD14 was upregulated in Rac1 mouse lesions. Rac1 dependent localization of CARD14 and IFIH1 was also demonstrated in PHKC. IFIH1, previously implicated in psoriasis GWAS promoted activation of NFκB and IRFs. The Rac1 dependent increase in both phosphorylated STAT3 and acetylated p65 in mouse skin suggests involvement of p65 acetylation by STAT3, and our backcrossing to immunodeficient mice demonstrate that an intact immune system is required to maintain PSTAT3 in Rac1-activated skin.

Rac1's capability to bind, activate, and promote nuclear translocation of STAT3 likely explains Rac1 dependent STAT3 activation and nuclear localization seen in Rac1 mouse epidermis and HPKC. Differences in early and late Rac1 dependent STAT3 activation in PHKC following EGF, TNFα and IL17 treatments respectively, may be reflective of these multiple ways in which Rac1 can promote STAT3 activity. STAT3 activation has been demonstrated in psoriatic epidermis and epidermal overexpression of activated STAT3 produced psoriatic skin lesions in mice. Interestingly, despite strain and platform discrepancies, we found a significant overlapping DEG signature with the K5STAT3C model. Both models implicate keratinocyte-intrinsic signaling cascades leading to immunocyte recruitment. The K5STAT3C and K14AREG mouse models of psoriasis shared pathways enriched for STAT3-, immune cell-, IL22- and JAK signaling. However, unlike Rac1 mouse skin, STAT3 mouse skin required repeated application of 12-0-teradecanoylphorbol-13-acetate (TPA) or tape stripping to drive lesion development. It is possible that while STAT3 was expressed in the STAT3 mouse, it was only upon injury induced Rac1 activation that activated Rac1 could effectively transport it to the nucleus which in turn could have been a key factor driving lesion development.

Our results suggest activation of Rac1 led to more than two-fold reduction in ZNF570 protein, previously implicated in psoriasis. Loss of ZNF750 protein expression has been demonstrated to regulate epidermal differentiation and proliferation, and may be one of several pathways through which Rac1 activation perturbs keratinocyte homeostasis. TGFα upregulation, previously linked to human psoriatic hyperplasia (51) was another likely effector of Rac1 induced proliferation in mouse lesions. We demonstrate that elevated Rac1 activation is directly linked to exaggerated proliferation of psoriatic epidermis, but require immune-derived stimulus.

Effective translation of mouse models to in vivo human psoriasis models has proven challenging. Of four transgenic mouse psoriasis models, K5-Tie2, K14-AREG, K5-STAT3, K5-TGFβ1 which were previously studied and correlated with human disease, as well as another transgenic psoriasis model of Jun protein deletion, none have been extended to a human psoriasis xenograft model. Also, while imiquimod induction of typical psoriasis lesions has been studied in a mouse model, clinical correlation to psoriasis patients has been questioned. Our autologous PHKC/PBMC xenograft model (FIG. 7A, B) is, to our knowledge, the first which reproduces the psoriatic hyperplasia/inflammation seen with full thickness human psoriasis skin/PBMC xenografts while allowing genetic manipulation of epidermal cells prior to xenografting. Epidermal inhibition of Rac1 activation in this model through N17Rac1 overexpression in PKHC normalized proliferation and inflammation in treated xenografts of human psoriasis tissue. Thus the results derived from our in vivo model of human psoriasis correlate well with our both our observations of human psoriasis tissue, as well as our Rac1 mouse model. In total these results suggest that epidermal Rac1 plays a critical role in facilitating the development of a feedback loop between the epidermis and immune system, promoting both the inflammatory and proliferative phenotype of psoriasis. Further, we delineate that suppression of immune derived factors or epidermal Rac1 activation present two distinct pathways for modulating aberrant Rac1 signaling. These findings implicate epidermal Rac1 as a novel target for psoriasis therapy.

### Materials and Methods

Transgenic Mice.

V12 Rac1 transgenic mice were generated by injection of a Rac1-cDNA construct into the pronucleus of fertilized oocytes. Founder mouse strain (CBA/CaJ, Jackson Labs) was backcrossed to both C57Bl6 and BALB/c (Jackson Labs) backgrounds and after five generations was observed to retain the same psoriatic phenotype as the founder strain. Rac1 c-DNA containing a point mutation C17 to A and a C-terminal Myc-tag (see Kjoller et al. JBC 152(6):1145-1157) was inserted via SacI-XbaI sites into a keratin 14 expression cassette harboring an SV40 intron and an SV40 polyA signal sequence at the N-terminus. Correct insertion was confirmed by direct DNA sequencing. After digestion of the plasmid with BssH2 the transgene was separated by agarose gel electrophoresis and isolated from the gel using the MinElute gel extraction kit (Qiagen, Hilden, Germany). Purification was performed using an Elutip mini column (Schleicher & Schüll, Dassel, Germany) and subsequent precipitation with ethanol. For pronucleus injection, the DNA was dissolved in microinjection buffer and adjusted to a final concentration of 10 ng/ml. Transgenic mice were generated by injection of the DNA construct into the pronucleus of fertilized oocytes. For screening of transgene insertion, genomic DNA was isolated from mouse tails and analyzed by means of polymerase chain reaction (PCR) using the primers SF3-25 5′-TTGGTTGTGTAACTGATCAGTAGGC-3′ and SF5-23 5′-TGGAGAGCTAGCAGGAAACTAGG-3′. Insertion was confirmed by Southern blot analysis using a 600-bp fragment as probe.

cDNA and siRNA constructs and vector information. V12 Rac1,V14 RhoA, dominant negative N17 Rac1 or LacZ control constructs were generated and cloned as previously described (Russell et al. (2003) J Cell Sci 116:3543-3556). Human V12 Rac1, N17Rac1 and LacZ constructs were a kind gift of Dr John Collard, Netherlands Cancer Institute, Amsterdam, The Netherlands. Human V14 RhoA was a kind gift from Dr Alan Hall, University College London, UK. ZNF750 was cloned into pLEX (Open Biosystems) with C-terminal FLAG, HA, and 6×HIS tags with the following primers: ZNF750 F: ACGCAGGATCCGCCACCATGAGTCTCCTCAAAGAGCGGAAGCCAAAAA; ZNF750 R: ACGCAGCGGCCGCGGGGACACCCGGGCCCTCCTTCGTAGTGTG.

Lentiviral gene transfer. 293T cells were transfected with 8 ug of lentiviral expression construct, 6 ug of pCMVD8.91, and 2 ug of pUCMD.G. Transfections were done in 10-cm plates using Lipofectamine 2000 (Life Technologies). Viral supernatant was collected 72 h after transfection and concentrated using a Lenti-X concentrator (Clontech). For ZNF750 experiments cells were after 48 hours transduced with pLEX control or pLEX ZNF750 lentivirus overnight.

Retroviral gene transfer. Phoenix cells were transfected with V12Rac1, V14 RhoA, N17 Rac1 or LacZ in 10-cm plates using Lipofectamine 2000 (Life Technologies). Cells were grown to 80% confluency, transferred to 32° C. incubation, and viral supernatant was collected after 24, 48 and 72 h. Cell cultures were incubated with polybrene for 10 minutes at 37° C., (5 ug/mL), media replaced by viral media with polybrene (5 ug/mL), centrifuged 1 hour at 1000 rpm, followed by incubation for 4 hours at 37° C., prior to media change.

SiRNA transduction and sequences. 1 million keratinocytes were electroporated with 1 nmol control or ZNF750 siRNA using Amaxa nucleofection reagents with siRNA sequences (control) GUAGAUUCAUAUUGUAAGGUU; (ZNF750): CCACCAGAGUUUCCACAUA′.

DNFB-induced contact allergic dermatitis and wounding models. For DNFB-induced contact allergic dermatitis, 1-Fluoro-2, 4-dinitrobenzene (Sigma) was diluted in acetone/olive oil (4:1). WT mice (n=3) were sensitized by painting 50 μl of 0.2% DNFB on the shaved abdomen on two consecutive days. Controls (n=3) were treated with 50 μl acetone-olive oil. For elicitation of contact allergic dermatitis, ears of mice were painted with 10 μl of 0.3% DNFB 10 days later, and harvested 24 hours after. For wounding assays, ears with 4 mm punch biopsies harvested, embedded in OCT, and 7 μm cryo-sections were analyzed 24, 48 and 72 hours after wounding.

Cyclosporin A injections. Seven day old Rac1-pups were treated daily IP with cyclosporine (15 mg/kg, n=3) or vehicle (n=3) for 21 days. Tail sections were harvested, embedded, and 7 μm cryo-sections fixed and stained. Average epidermal thickness (excluding stratum corneum) was measured across 4× 10× fields. Ki67 and CD3+ cells were quantified using Image J.

Joint Imaging. Rac1 and WT mice littermates at six weeks of age (n=6) where placed under isofluorane anesthesia, and scanned with the Gamma Medica eXplore CT 120 microCT scanner (GE healthcare) at the Stanford small animal imaging facility. The images were taken at 97 micrometer thickness, calibrated and 3D reconstructions were created using GE Microview software.

RNA extraction and RT-qPCR. RT-qPCR was performed using the Roche480 LightCycler with Maxima SYBR Green master mix (Fermentas), or SYBR Select Master Mix (Invitrogen). Samples were run in triplicates and normalized to 18S RNA.

Confocal microscopy. Tissue sections were embedded in OCT, snap frozen, and 7 μm sections cut on a cryostat (Leica), and sections or cells on coverslips were fixed for 10 minutes with cold methanol, washed with TBS, then blocked for one hour at room-temp with 10% normal goat, donkey or human serum, and incubated with primary antibody in PBS or TBS overnight. 12 hours later, washing was repeated 3 times 5 minutes, followed by incubation with secondary antibodies 1:400 together with Hoescht 1:5000 for 1 hour at room temperature washed and mounted with fluoromount (Southern Biotech). For mouse antibodies on mouse tissue, sections were treated with MOM igG blocking kit (Vector laboratories), according to manufacturer's recommendations.

Slides were imaged using confocal microscopy (LSM-700, Zeiss, Germany) and were processed and quantified using Image J. Rac1GTP (#26903, NewEast Biosciences), RhoAGTP (#26904, NewEast Biosciences), phospho-STAT3 (tyr705, d3a7xp, #9145, Cell Signaling), phospho-nfκb p65 (ser536,93h1) with forceps, epidermal sheets trypsinized for 15 min, neutralized with DMEM (Mediatech Inc) containing 10% FBS, 1% antibiotic-antimycotic (30-004-CI, Mediatech Inc), centrifuged for 5 min at 1000 rpm, and re-suspended in a 50-50 mixture of supplemented (Human Keratinocytes Growth Supplement, S-001-5, Invitrogen) medium 154 (M16 254-500, Invitrogen) and K-SFM (Defined Keratinocyte SFM, 10744-019, Invitrogen) and 1% antibiotic-antimycotic solution (0-004-CI, Mediatech Inc). Adult cells were at passage 3 prior to in vitro analysis or xenografted.

SRPG-1 peptidoglycan stimulation. Primary human keratinocytes (n=3) from non-lesional psoriatic (n=3) or healthy control (n=3) skin were isolated from skin biopsies through dispase treatment as previously described, and cultured on collagen-coated coverslips in 6-well plates. Cells were attached for 4 hours, growth factor starved for 24 hours and stimulated with 1 ug/ml-1 SRPG-1 (#SRPG-1, lot 112503SRPG, Toxin Technologies, Saratoga, Fla., US) for 10 or 90 minutes.

Cytokine stimulation. Primary human adult psoriatic or adult normal control keratinocytes were growth factor starved for 24 hours then stimulated using 5 or 50 ng/ml EGF (PHG0311, Life Technologies), 100 ng/ml TNFα, (PHC3016, Gibco), 25 ng/ml IL22 (NBP1-99226, Novus) or 100 ng/ml IL17A/F (P4799, Novus Biologicals) and harvested after 0, 10 and 90 minutes; or for II17A/F experiments GF starved or stimulated for 24 hours.

MTT assay. 5000 first-passage neonatal keratinocytes per well were seeded on a collagen-coated 96 well plate, and incubated with 50/50 (Medium 154 and Keratinocyte—SFM medium, Invitrogen with Human Keratinocytes Growth Supplement, S-001-5, Invitrogen, and medium 154 supplement M-154-500, Invitrogen) with 1% antibiotic-antimycotic (30-004-CI, Mediatech Inc) for 48 hours. 10 ul MTT reagent (MTT proliferation assay, ATCC, Manassas, US) was added for 4 hours, 100 ul detergent was added to each well for 2 hours, and absorbance read at 570 nm (Spectramax M5, Molecular Devices, US), normalized to cell-free control absorbance.

Keratinocyte differentiation assay. 36 hours after transduction, keratinocytes were plated at 40 k in 6-well plates for the undifferentiated condition or 400 k in 12-well plates for the differentiated condition. Each condition was in triplicate. After 16 hours, 1.2 mM calcium was added to the differentiated condition. After 3 days, RNA was extracted using a RNeasy plus kit (Qiagen) or cells lysed using 1× cell lysis solution (Thermo Scientific) with 1% halt proteinase-phosphatase inhibitor (Thermo Scientific).

Immunoblot and pulldown assays Tissue or cells were lysed in 1× cell lysis solution (Thermo Scientific) with 1% Halt proteinasephosphatase inhibitor (Thermo Scientific). For Rac1GTP pulldown, the ratio of active and total Rac1, respectively, were quantified using an Active Rac1 Pull-Down and Detection Kit, Thermoscientific, US, according to manufacturer's recommendations. Quantification of immunoblots in biologic triplicates or Rac1GTP pulldown in duplicates (IL22), triplicates (IL17A/F, TNFα) or quadruplicates (EGF) was performed by densitometry in Image J.

Genome-wide transcriptional analysis. Whole-skin from day 7 Rac1 pups or normal skin from wildtype littermates (n=6) were analyzed using Illumine MEEBO arrays, and compared to a dataset of 214 lesional psoriasis skin samples and 85 non-psoriasis skin samples, or gene expression datasets of skin from the KSSTAT3C, K14AREG, K5Tie2, K5-TGFβ1 and IMQ mouse models of psoriasis.

Luminex assays. For human cells Rac1 GTP or LacZ overexpressing primary human keratinocytes (PHKCs), primary human keratinocytes from non-lesional psoriatic skin retrovirally overexpressing dominant negative Rac1 (N17) or LacZ (paired) or PBMCs from healthy donors (derived from the Stanford Blood Center and isolated from buffy-coat according to standard procedures) were plated in equal density on collagen-coated 6 well plates. After 48 hours in 2 ml KGM either alone (80 000 KCs) or for each keratinocyte condition in co-culture with PBMCs (ratio 1:10), supernatants were centrifuged at 1000 rpm to pellet residual cells. Samples were run in duplicates. For mouse serum analysis, three week old Rac1 (n=3) or WT littermates (n=3) were harvested and serum isolated by centrifugation. Human 51-plex or mouse 38-plex luminex assays were performed in the Human Immune Monitoring Center at Stanford University.

Human xenografts Primary keratinocytes and fibroblasts were isolated from 4 mm punch biopsies of human control or human psoriatic non-lesional skin, through dispase treatment (Fisher, 30 U/ml, 4° C. ON) and trypLE digestion (Invitrogen, 15 min, 37° C.), seeded on devitalized dermis and grown at the airfluid interphase for 7 days prior to being xenografted to NOD/SCID mice. Autologous PBMCs were injected intradermally and grafts harvested after 14 days. Keratinocytes were grown in supplemented 50/50 medium-154 and defined keratinocyte SFM as described, and fibroblasts in DMEM (Mediatech Inc), with 10% FBS. 0.5×10⁶ fibroblasts were centrifuged (2×20 min, 1000 rpm) onto reticular side of a 10×10 mm²), with minor modifications. 35 mm plastic inserts were prepared with an 8 mm² square central orifice resting on anchored 3 mm glass beads inside a 60 mm plastic tissue culture dish. Reticular side of skin equivalent was covered in matrigel (BD Biosciences), dried for 5 minutes and flipped onto 35 mm insert, covering an 8 mm² orifice, exposing papillary side of skin equivalent to the air-fluid interphase. 1×10⁶ LacZ or N17 Rac1 keratinocytes were seeded in 100 ul 50/50 M-154/defined keratinocyte SFM media into orifice on papillary side, settled for 10 minutes, and 5 ml of KGM pipetted into lower chamber comprising of a 60 mm tissue culture dish. Media was changed in lower chamber daily for 7 days. On day 8, skin equivalents containing psoriatic keratinocytes and autologous fibroblasts (LacZ n=4 and N17 n=2) or control keratinocytes (LacZ n=4) and autologous fibroblasts were grafted onto 8 week old NOD/SCID male mice (Jackson Labs), sutured and bandaged. After ten days, bandages and sutures were removed. The following day, blood samples were obtained from each subject, and PBMCs were isolated using Ficoll-paque plus per manufacturer's recommendations (GE Healthcare). 150 ul of RPMI or RPMI with PBMCs (1×10⁶) were subsequently injected intradermally into LacZ-psoriasis (PBMCs n=2, RPMI n=2), N17 psoriasis (PBMCs n=2) or LacZ-control xenografts (PBMCs n=2, RPMI n=2). Samples were harvested after 14 days. 35 mm plastic inserts were prepared with an 8 mm² square central orifice resting on anchored 3 mm glass beads inside a 60 mm plastic tissue culture dish. Reticular side of skin equivalent was covered in matrigel (BD Biosciences), dried for 5 minutes and flipped onto 35 mm insert, covering an 8 mm² orifice, exposing papillary side of skin equivalent to the air-fluid interphase. 1×10⁶ keratinocytes were seeded in 100 ul 50/50 M-154/defined keratinocyte SFM media into orifice on papillary side, settled for 10 minutes, and 5 ml of KGM pipetted into lower chamber comprising of a 60 mm tissue culture dish. Media was changed in lower chamber daily for 7 days, then harvested, embedded in OCT, and snap-frozen.

Statistical analysis. Human tissue analysis was performed on 19 patients and 10 normal controls, in vitro and xenograft studies were performed on 2-7 patient donors and 3-5 controls. For transgenic mice studies groups contained 3-6 animals per group. For transgenic mouse studies, mice were randomized, and for all experiments analysis blinded. No outliers were removed from final analysis. In all three model systems, numbers and replicates are outlined in material and methods or figure legends. Unpaired t-tests with Welch's correction and Mann-Whitney/Wilcoxon ranked-sum tests or Tukey's multiple comparison tests with correction for multiple testing were performed to compare mean values between experimental groups using Graphpad Prism software. All tests were two-tailed unless specified. Differential gene expression using ANOVA was performed using Partek Genomics Suite 6.6 and resulting p-values were corrected for multiple-hypothesis testing using the Benjamini or Bonferroni method using DAVID and KEGG pathway analysis or the Protein ANalysis THrough Evolutionary Relationships database, and non-significant predictions in IPA were filtered using Fisher's exact test. For gene-set comparisons between groups, significance was determined using a hypergeometric test. p-values under 0.05 were considered significant and corrected for multiple testing where applicable.

Study approval. Mouse studies were approved and in agreement with Stanford University IACUC institutional guidelines, Assurance number: A3213-01 protocol ID: 10364. Human studies were conducted according to Declaration of Helsinki principles, in agreement with approved human subject protocol, assurance number: FWA00000935 (SU), FWA00000934 (SHC). Protocol ID: 12047, IRB: 4593 (Panel: 5). Informed consent was obtained.

RNA isolation and primer sequences for RT-qPCR. RNA was extracted from cell lysates; or mouse skin from day 1 or day 7 Rac1 or wild type pup skin using a Qiagen RNA plus miniprep kit. RNA concentration was determined with spectrophotometric analysis; purity analyzed with 260:280 absorbance ratios. One ug of RNA was reverse-transcribed using iSCRIPT cDNA synthesis kit (Bio-Rad) or High Capacity RNA-to-cDNA Kit (Invitrogen).

Human qPCR primer sequences.
18S F:
GCAATTATTCCCCATGAACG;
18S R:
GGCCTCACTAAACCATCCAA;
ZNF750 F:
AGCTCGCCTGAGTGTGAC;
ZNF750 R:
TGCAGACTCTGGCCTGTA;
LCE3D F:
GCTGCTTCCTGAACCAC;
LCE3D R:
GGGAACTCATGCATCAAG;
SPRR2G F:
GGACTCTCCACCACACTGATG;
SPRR2G R:
CTGCTGCTGCTGGTAAGACAT;
SPRR3 F:
CCAGGCTACACAAAGCTAC;
SPRR3 R:
GCTTAATTCAGGGGCTTAC;
IVL F:
AAAGCACCTAGAGCACCC;
IVL R:
GGTTGAATGTCTTGGACCT;
LOR F:
CTCTGTCTGCGGCTACTCTG;
LOR R:
CACGAGGTCTGAGTGACCTG.
Mouse qPCR primer sequences.
CCL2:
Mm00441242_m1 (Thermo Scientific);
CCL5 F:
GCAAGTGCTCCAATCTTGCA;
CCL5 R:
CTTCTCTGGGTTGGCACACA;
CCL5 Probe:
TGTTTGTCACTCGAAGGAACCGCCA;
CCL20:
Mm00444228_m1 (Thermo Scientific);
CXCL1:
Mm00433859_m1 (Thermo Scientific);
CXCL2:
Mm00436450_m1 (Thermo Scientific);
CXCL10:
Mm00445235_m1 (Thermo Scientific);
Cxcl11:
Mm00444662_m1 (Thermo Scientific);
β4 Defensin F:
TGGTGCTGCTGTCTCCACTTGC;
β4 Defensin R:
CGAAAAGCGGTAGGGCACGGA.
CCL17 F:
GCCTCTCGTACATACAGACGC;
CCL17 R:
CCAGTTCTGCTTTGGATCAGC;
CCL20 F:
TACCATGAGGTCACTTCAGATGC;
CCL20 R:
GCACTCTCGGCCTACATTGG;
IL17RE F:
CAGTCCCAGTGACGCTAGAC;
IL17RE R:
ACCCACTAGAGCGGTGAGAG;
TSLP F:
ACGGATGGGGCTAACTTACAA;
TSLP R:
AGTCCTCGATTTGCTCGAACT;
OSMR F:
GCATCCCGAAGCGAAGTCTT;
OSMR R:
GGGCTGGGACAGTCCATTCTA;
CCL5 F:
GCTGCTTTGCCTACCTCTCC;
CCL5 R:
TCGAGTGACAAACACGACTGC;
CCR6 F:
TGGGCCATGCTCCCTAGAA;
CCR6 R:
GGTGAGGACAAAGAGTATGTCTG;
IL23P19 F:
CAGCAGCTCTCTCGGAATCTC;
IL23P19 R:
TGGATACGGGGCACATTATTTTT;
IL1β F:
GAAATGCCACCTTTTGAC ACT G;
IL1β R:
TGGATGCTCTCATCAGGACAG;
IL1F6 F:
GCAGCATCACCTTCGCTTAGA;
IL1F6 R:
CAGATATTGGCATGGGAGCAAG

Immunoblot assays. Cells or tissue was lysed in 1× cell lysis solution (Thermo Scientific) with 1% Halt proteinase-phosphatase inhibitor (Thermo Scientific), incubated at 4° C. under rotation for 1 hour and centrifuged 15 minutes for 13200 rpm at 4° C. Lysates were quantified based on absorbance with a Bradford assay, using standard conditions.

Lysates were denatured in 100° C. for 5 minutes with 4×NUPAGE sample loading buffer (Invitrogen), 10×NUPAGE sample reducing agent (Invitrogen), and 5% p mercaptoethanol. Subsequently, lysates were loaded on a 4-12% bis-tris gel with 1×MOPS running buffer and run for 90 min at 150V. Gels were transferred with 1× transfer buffer in 10% methanol for 2.5 hours at 25V. Membranes were stained with Ponceau red, prior to being blocked (5% milk or 5% or 3% BSA), washed and incubated with primary antibody in 3% BSA overnight, washed and incubated with a HRP-tagged secondary antibody for 1 hour at RT in 2% BSA or 5% milk, washed and developed.

### Example 2

### Benzamil Reverses Psoriasiform Hyperplasia In Vivo Animal Model

We utilized the Rac1_{V12} transgenic mouse model of psoriasis for in vivo validation of treatment with benzamil. We administered benzamil or saline control systemically through intreaperitoneal injections (EOD 20 injections). Benzamil treated mice exhibited marked signs of improvement, including reduced scaling, erythema and edema on muzzle and ears and healing of tail lesions. Treated skin exhibited reduced epidermal hyperproliferation, in a dose dependent fashion, and was accompanied by reduced epidermal Ki67, TGFα, phospho-STAT3, phospho-relA and reduced CD3+ skin infiltrating T cells. We found reduced mRNA expression of both keratin 16, s100a7 and tnfa by RT-qPCR. In agreement with our in vitro assays, we found reduced Rac1_{GTP} in skin of treated mice compared to vehicle control, comparable to wildtype mice skin outside of K14₊ layers.

To control benzamil release over time, Rac1 V12 mice were also implanted osmotic pumps containing benzamil or vehicle control for 28 days. Constant release rate of benzamil significantly reduced epidermal thickness compared to vehicle, despite a 30% shorter treatment duration than IP injected mice. Whole skin lysates from mice treated with benzamil or vehicle showed normalized NFκB as reduced STAT3 signaling. This was accompanied by significantly reduced suprabasal proliferation, and CD3+ skin infiltrating cells. Thus, these results experimentally validates in vivo that benzamil reverses psoriasiform hyperplasia and signaling.

1. An animal model for psoriasis, in which keratinocytes have been modified to express activated Rac1, and where the animal mimics the phenotype of human psoriasis.

2. The animal model of claim 1, wherein the animal is a transgenic mouse.

3. The animal model of claim 1, wherein the activated Rac1 is V12 Rac1.

4. The animal model of claim 2, wherein the activated Rac1 is operably linked to a keratinocyte promoter.

5. The animal model of claim 1, wherein activated Rac1 is expressed in human xenografted keratinocytes.

6. The animal model of claim 5, wherein the activated Rac1 is endogenous.

7. The animal model of claim 5, wherein the keratinocytes are genetically modified to express an activated form of Rac1.

8. The animal model of claim 5, wherein the animal model further comprises fibroblasts and peripheral blood mononuclear cells autologous to the keratinocytes.

9. A method of screening a candidate agent for activity in the treatment of psoriasis, the method comprising:
contacting an animal model of claim 1 or cells derived therefrom with a candidate agent suspected of inhibiting activated Rac1; and
determining the effect on Rac1 activation.

10. The method of claim 9, wherein the effect on Rac1 activation is determination in epidermal cells.

11. The method of claim 9, wherein the contacting is topical.

12. The method of claim 9, wherein the effect on a psoriasis phenotype is determined.
